(19) United States
(12) Patent Application Publication
(10) Pub. No.: US 2021/0246886 A1
(43) Pub. Date: Aug. 12, 2021

(54) Fluorosulfones

(71) Applicant: 3M INNOVATIVE PROPERTIES COMPANY, St. Paul, MN (US)

(72) Inventors: William M. Lamanna, Stillwater, MN (US); Sean M. Smith, Woodbury, MN (US); Michael G. Costello, Afton, MN (US); Michael J. Bulinski, Stillwater, MN (US); John G. Owens, Woodbury, MN (US); Markus E. Hirschberg, Muhldorf (DE); Klaus Hintzer, Kastl (DE); Bamidele O. Fayemi, Cottage Grove, MN (US); Philip E. Tuma, Faribault, MN (US); Nicholas S. Johnson, Minneapolis, MN (US); Forrest A. Coughlin, Chaska, MN (US); Jay R. Nierengarten, Woodbury, MN (US)

(21) Appl. No.: 17/049,752

(22) PCT Filed: Apr. 23, 2019
(86) PCT No.: PCT/IB2019/053347

§ 371 Date: Oct. 22, 2020

Related U.S. Application Data
(60) Provisional application No. 62/662,980, filed on Apr. 26, 2018.

(51) Int. Cl. F03G 7/00; C07C 317/04; F25D 3/00

(52) U.S. Cl. CPC F03G 7/00; F25D 3/00; C07C 317/04

(57) ABSTRACT

A foamable composition includes a blowing agent, a foamable polymer or a precursor composition thereof, and a nucleating agent. The nucleating agent includes a compound having structural formula (I) R¹SO₂R²(SO₂R³)ₙ (I) where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

### FIELD

The present disclosure relates to fluorosulfones and methods of making and using the same, and to working fluids that include the same.

### BACKGROUND

Various fluorosulfones are described in, for example, UK Patent No. 1,189,561, U.S. Pat. Nos. 6,580,006, and 7,087,788.

### SUMMARY

In some embodiments, a foamable composition is provided. The foamable composition includes a blowing agent, a foamable polymer or a precursor composition thereof, and a nucleating agent. The nucleating agent includes a sulfone having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

In some embodiments, a device is provided. The device includes a dielectric fluid comprising a compound having the above-described structural formula (I). The device is an electrical device.

In some embodiments, an apparatus for converting thermal energy into mechanical energy in a Rankine cycle is provided. The apparatus includes a working fluid, a heat source to vaporize the working fluid and form a vaporized working fluid, a turbine through which the vaporized working fluid is passed thereby converting thermal energy into mechanical energy, a condenser to cool the vaporized working fluid after it is passed through the turbine, and

a pump to recirculate the working fluid. The working fluid comprises a compound having the above-described structural formula (I).

In some embodiments, an immersion cooling system includes a housing having an interior space, a heat-generating component disposed within the interior space, and a working fluid liquid disposed within the interior space such that the heat-generating component is in contact with the working fluid liquid. The working fluid includes a compound having the above-described structural formula (I).

In some embodiments, a thermal management system for a lithium-ion battery pack includes a lithium-ion battery pack, and a working fluid in thermal communication with the lithium-ion battery pack. The working fluid includes a compound having the above-described structural formula (I).

In some embodiments, a thermal management system for an electronic device is provided. The thermal management system includes an electronic device selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, or a laser. The thermal management system further includes a working fluid in thermal communication with the electronic device. The working fluid includes a compound having the above-described structural formula (I).

In some embodiments, a system for making reactive metal or reactive metal alloy parts is provided. The system includes a molten reactive metal selected from magnesium, aluminum, lithium, calcium, strontium, and their alloys. The system further includes a cover gas disposed on or over a surface of the molten reactive metal or reactive metal alloy. The cover gas includes a compound having the above-described structural formula (I). The compound of structural formula (I) has a GWP (100 year ITH) of less than 2000.

The above summary of the present disclosure is not intended to describe each embodiment of the present disclosure. The details of one or more embodiments of the disclosure are also set forth in the description below. Other features, objects, and advantages of the disclosure will be apparent from the description and from the claims.

### BRIEF DESCRIPTION OF THE FIGURES

FIG. 1 is a schematic of a two-phase immersion cooling system in accordance with some embodiments of the present disclosure.

FIG. 2 is a plot of the heat transfer coefficient of an embodiment of the present invention and a comparative example.

FIG. 3 is a schematic of a Rankine cycle.

FIG. 4 shows 2P Lithium-ion batteries with nail puncture and fluid application points.

FIG. 5 shows the mean temperature in the adjacent cells in battery thermal runaway prevention testing for a fluid flow rate of 50 mL/min for one minute after puncture of the initial cell.

FIG. 6 shows the mean temperatures in the adjacent cells in battery thermal runaway prevention testing for a fluid flow rate of 25 mL/min for two minutes after initial cell puncture.

FIG. 7 shows the temperatures of the initial cell and the adjacent cell temperatures in battery thermal runaway prevention testing for a fluid flow rate of 50 mL/min for one minute after initial cell puncture.

FIG. 8 shows the temperatures of the initial cell and the adjacent cell temperatures in battery thermal runaway prevention testing for a fluid flow rate of 25 mL/min for two minutes after initial cell puncture.

FIG. 9 is a plot of cell size distributions of foams prepared with and without fluorosulfone additive of the present invention.

### DETAILED DESCRIPTION

Specialty materials, such as sulfur hexafluoride (SF₆), perfluorocarbons (PFCs), perfluorinated tertiary-amines (PFAs), perfluoropolyethers (PFPEs) and hydrofluorocarbons (HFCs), have combinations of properties that make them useful in applications such as, for example, electrical power generation and transmission, reactive metal casting, heat transfer for thermal management in electronic devices and batteries, thermal runaway protection for batteries, heat transfer in semi-conductor manufacturing, semiconductor etching and cleaning, and for use as foam blowing additives. These specialty materials generally have low flammability or are nonflammable, have very good thermal and chemical stability, are generally low in toxicity, are not ozone depleting, and in addition have properties needed for the applications, such as low electrical conductivity, high dielectric strength, high heat capacity, high heat of vaporization, high volatility, very low residue after drying, noncorrosive and low mutual solubility in organics.

The good thermal and chemical stability of SF₆, PFCs, PFPEs, and HFCs also translates into long atmospheric lifetimes and high global warming potentials (GWPs). As a result, some of these materials are included in the list of greenhouse gases, which were subject to the Kyoto Protocol and subsequent regulations to control emissions. The objective of these regulations is to reduce the emission of greenhouse gases from processes using greenhouse gases and to reduce or minimize their impact on climate change. Capture of emissions and/or destroying them before emission has proven to be both difficult and costly. Replacement materials with more environmentally acceptable properties are needed for these applications.

Two groups of advanced materials, hydrofluoroethers (HFEs) and fluoroketones (FKs), have been shown to satisfactorily replace high GWP materials in a few applications such as fire extinguishing agents and precision cleaning and coating of electronics and in processes used to manufacture them. However, these materials cannot act as replacements in all applications due to chemical stability limitations. In some applications, HFE and FK chemical compositions are not suitable. For example, the carbon backbone of HFEs are likely to form conducting carbonaceous deposits if used as a dielectric insulating gas in power transmission equipment and cause equipment failure. And, for use as polyurethane foam blowing additives, HFEs and FKs are generally too reactive with the polyol/amine components of the foam formulation to be useful.

As a result, additional substitute materials are desired that will perform satisfactorily and safely in certain applications. These new substitute materials also should have much shorter atmospheric lifetimes and lower GWPs compared to the materials they replace to be environmentally acceptable.

Fluorosulfones of the present disclosure have many of the properties that are desired for application in, for example, insulating dielectric gases for electrical power generation and transmission, protective cover agents for reactive molten metal casting, direct contact immersion cooling and heat transfer, semiconductor etching and cleaning, working fluids for organic Rankine cycle equipment, and for use as foam blowing additives. Generally, fluorosulfones of the present disclosure are electrically non-conducting, nonflammable (i.e. no flashpoint as measured by ASTM D-3278-96 “Standard Test Methods for Flash Point of Liquids by Small Scale Closed-Cup Apparatus” or ASTM method D 7236-06 “Standard Test Method for Flash Point by Small Scale Closed Cup Tester” (Ramp Method)), and have good thermal properties for use as working fluids in certain heat transfer processes. Certain fluorosulfones of the present disclosure are low boiling or gaseous for applications requiring higher volatility, such as insulating dielectric gases. Others are less volatile with boiling points suitable for use in direct contact immersion cooling or as working fluids for organic Rankine cycle equipment to convert otherwise wasted heat to electricity. Fluorosulfones of the present disclosure exhibit high chemical stability in the presence of certain reactive compounds allowing them to be used, for example, in processes that include reactive amine bases and alcohols commonly employed in the production of polyurethane foams.

Certain fluorosulfones, particularly perfluorosulfones, have been described as having high chemical and thermal stability. Historically, high chemical and thermal stability have been shown to translate into long atmospheric lifetimes and high GWPs, making materials with such characteristics unsuitable for many emissive applications.

Surprisingly, however, it has been discovered that fluorosulfones of the present disclosure, including perfluorosulfones, are reactive towards hydroxyl radicals and undergo degradation in the troposphere so their atmospheric lifetime is significantly less than SF₆, perfluorocarbons (PFCs), perfluorinated amines (PFAs), perfluoropolyethers (PFPEs), and most hydrofluorocarbons (HFCs). This reduces their GWP and their contribution as greenhouse gases to acceptable levels.

While fluorosulfones of the present disclosure have good chemical stability under normal use conditions, exposure to hydroxyl radicals causes the materials to break down. Even perfluorosulfones of the present disclosure, with completely fluorinated (perfluorinated) carbon backbones, have been found to be surprisingly reactive towards hydroxyl radicals in atmospheric chamber experiments designed to mimic the troposphere. As a result, perfluorosulfones of the present disclosure have been found to have much shorter atmospheric lifetimes than was previously expected. The surprisingly rapid atmospheric destruction of perfluorosulfones of the present disclosure reduces their expectedly long atmospheric lifetimes such that they are much lower than many other perfluorinated materials (e.g., PFCs, PFAs, PFPEs) and renders them much more environmentally acceptable in several applications where there is need for replacement of high GWP materials.

Perfluorinated sulfones have been reported to react readily with a variety of nucleophiles, including oxygen and nitrogen centered nucleophiles, as described in J. Fluorine Chemistry, 117, 2002, pp 13-16. Studies suggest that susceptibility to nucleophilic attack can be correlated with elevated toxicity for certain families of fluorochemicals, as described in J. Fluorine Chemistry, 125, 2004, pp 685-693, and Chem. Res. Toxicol., 27(1), 2014, pp 42-50. Therefore, conventional wisdom suggested that the pronounced reactivity of perfluorosulfones toward nucleophilic attack would similarly lead to elevated toxicity. However, perfluorosulfones of the present disclosure have surprisingly been found to exhibit very low toxicity based on standard acute 4-hour inhalation toxicity tests in rats at relatively high doses (displaying LC-50s greater than 10,000 ppm or greater than 20,000 ppm).

Similarly, conventional wisdom suggested that the reported susceptibility of perfluorosulfones to nucleophilic attack would make them unsuitable for use in applications where they are exposed to nucleophilic reagents for extended periods of time. Yet, perfluorosulfones of the present disclosure have shown surprising stability in the presence of standard polyol/amine catalyst mixtures commonly used in the production of polyurethane foams and known to undergo destructive nucleophilic attack with other reactive foam additives. As a result, these perfluorosulfones have shown unexpected utility as stable foam additives (nucleating agents) for reducing cell size in blown polyurethane foams, a critical parameter in optimizing the insulating properties of such foams.

Still further, perfluorosulfones of the present disclosure have been found to provide exceptionally high dielectric breakdown strengths in the gas phase when compared to other common perfluorinated materials at equivalent pressures in the gas phase, such as perfluoropropane (C₃F₈), perfluoro-cyclo-propane (cyclo-C₃F₆), and even the widely used perfluorinated dielectric gas, sulfur hexafluoride (SF₆). The unexpectedly high gas phase dielectric breakdown strengths of the perfluorosulfones of the present disclosure stands in surprising contrast to their inferior dielectric strength in the liquid phase compared to perfluorinated fluids like FC-3283 (a PFA) and Galden HT-110 (a PFPE) and FC-72 (a PFC available from 3M, St. Paul, Minn.). This, along with their surprisingly low GWPs compared to other perfluorinated materials makes them well suited for applications where an insulating dielectric gas is needed to prevent dielectric breakdown and arcing without significant adverse environmental effects. Thus, perfluorosulfones of the present disclosure are attractive candidates for SF₆ replacement in medium to high voltage switch gear and high voltage gas insulated power lines, for example, to achieve insulating dielectric performance comparable to or better than SF₆, while also providing significantly improved environmental sustainability.

Yet another area where perfluorosulfones of the present disclosure have shown surprising utility is in immersion cooling and thermal management applications, including but not limited to direct contact single-phase and two-phase immersion cooling and thermal management of electronic devices and batteries. These applications generally impose a long list of necessary requirements on the fluids employed, including non-flammability, low toxicity, low GWP, excellent dielectric properties (i.e., low dielectric constant, high dielectric strength, high volume resistivity), long term thermal and hydrolytic stability, and good low temperature properties (low pour point and low viscosity at low temperatures). In two-phase immersion cooling applications, suitable fluids should also have a boiling point in the right range for the intended application and a high heat of vaporization. It can be extremely difficult to meet all these requirements. Existing materials that are used today in immersion cooling and thermal management applications include HFEs, PFCs, PFPEs, PFAs, and PFKs. All have utility in certain applications but none provide universal utility due to one or more deficiencies. The PFCs, PFPEs and PFAs have very high global warming potentials, typically exceeding 8000 (100 year ITH), leading to environmental concerns in emissive applications. The HFEs have relatively high dielectric constants and are thus not compatible with electronic equipment operating at high signal frequencies due to detrimental effects on signal integrity. The PFCs, PFPEs, PFAs, PFKs, and HFEs have relatively low heats of vaporization for use in two-phase immersion applications, which has a negative impact on cooling efficiency. Some PFKs can have limited hydrolytic stability under certain extreme conditions, which can result in gradual hydrolysis over extended periods. Perfluorosulfones of the present disclosure overcome many of the issues and shortcomings of existing materials. For example, perfluorosulfones of the present disclosure provide much lower GWPs than PFCs, PFPEs, and PFAs. Perfluorosulfones of the present disclosure also provide significantly lower dielectric constants than the HFEs. In addition, perfluorosulfones of the present disclosure provide improved hydrolytic stability compared to PFKs and HFEs. And perfluorosulfones of the present disclosure generally provide higher heats of vaporization compared to HFEs, PFKs, PFCs, PFPEs and PFAs, for improved two-phase immersion cooling efficiency. Thus, the perfluorosulfones of the present disclosure provide a superior balance of properties for use in direct contact immersion cooling and thermal management applications than many materials on the market today, while also providing non-flammability and low toxicity.

As used herein, “catenated heteroatom” means an atom other than carbon (for example, oxygen, nitrogen, or sulfur) that is bonded to at least two carbon atoms in a carbon chain (linear or branched or within a ring) so as to form a carbon-heteroatom-carbon linkage.

As used herein, “fluoro-” (for example, in reference to a group or moiety, such as in the case of “fluoroalkylene” or “fluoroalkyl” or “fluorocarbon”) or “fluorinated” means (i) partially fluorinated such that there is at least one carbon-bonded hydrogen atom, or (ii) perfluorinated.

As used herein, “perfluoro-” (for example, in reference to a group or moiety, such as in the case of “perfluoroalkylene” or “perfluoroalkyl” or “perfluorocarbon”) or “perfluorinated” means completely fluorinated such that, except as may be otherwise indicated, there are no carbon-bonded hydrogen atoms replaceable with fluorine.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure concerns fluorosulfones represented by the following general formula:

R¹SO₂R²(SO₂R³)ₙ

where R¹, R², and R³ are independently a fluoroalkyl group having from 1 to 10 carbon atoms (from 1 to 5 carbon atoms, 1 to 3 carbon atoms, 1 to 2 carbon atoms, 4 to 8 carbon atoms, 2 to 5 carbon atoms, or 1 carbon atom) that is linear, branched, or cyclic and optionally contains at least one catenated ether oxygen atom or a trivalent nitrogen atom, and n is 0 or 1. In some embodiments, when n is 1, R² is a fluoroalkylene group; and in some embodiments, when n is 0, le and R² can be linked together to form a ring structure. The carbons on the fluoroalkyl groups (R¹, R², and R³) may contain fluorine atoms and/or fluorine and hydrogen atoms. When any or all of the fluoroalkyl groups contain hydrogen, the ratio of fluorine to hydrogen in the molecule is sufficient such that there is no flash point as measured by ASTM D-3278-“Standard Test Methods for Flash Point of Liquids by Small Scale Closed-Cup Apparatus” or ASTM method D 7236-06 “Standard Test Method for Flash Point by Small Scale Closed Cup Tester” (Ramp Method). In some embodiments, any or all of R¹, R², and R³ are perfluorinated alkyl groups and thus contain no hydrogen atoms bound to carbon. In some embodiments, n is 0 and R¹ and R² are not linked together to form a ring structure.

Representative examples of the fluorosulfones of the present disclosure include but are not limited to the following:

CF₃SO₂CF₃, CF₃SO₂C₂F₅, CF₃SO₂CF(CF₃)₂, CF₃SO₂C₃F₇, CF₃SO₂CF(CF₃)CF₂CF₃, CF₃SO₂CF₂CF(CF₃)₂, CF₃SO₂C₄F₉, CF₃SO₂CF(CF₃)OCF₃, CF₃SO₂CF(CF₃)OC₃F₇, CF₃SO₂CF(CF₃)OCF₂CF(CF₃)OC₃F₇, C₂F₅SO₂C₂F₅, C₂F₅SO₂CF(CF₃)₂, C₂F₅SO₂C₃F₇, C₂F₅SO₂C₄F₉, C₂F₅SO₂CF(CF₃)CF₂CF₃, C₂F₅SO₂CF₂CF(CF₃)₂, C₂F₅SO₂CF(CF₃)OCF₃, C₂F₅SO₂CF(CF₃)OC₃F₇, C₂F₅SO₂CF(CF₃)OCF₂CF(CF₃)OC₃F₇, C₃F₇SO₂CF(CF₃)₂, C₃F₇SO₂CF(CF₃)₂, C₃F₇SO₂C₃F₇, C₃F₇SO₂C₄F₉, C₃F₇SO₂CF(CF₃)CF₂CF₃, C₃F₇SO₂CF₂CF(CF₃)₂, C₃F₇SO₂CF(CF₃)OCF₃, C₃F₇SO₂CF(CF₃)OC₃F₇, C₃F₇SO₂CF(CF₃)OCF₂CF(CF₃)OC₃F₇, C₄F₉SO₂CF(CF₃)₂, C₄F₉SO₂C₄F₉, C₄F₉SO₂CF(CF₃)CF₂CF₃, C₄F₉SO₂CF(CF₃)OCF₃, C₄F₉SO₂CF(CF₃)OC₃F₇, C₄F₉SO₂CF(CF₃)OCF₂CF(CF₃)OC₃F₇, (CF₃)₂CFSO₂CF₂SO₂CF(CF₃)₂, CF₃CF(OCF₃)SO₂CF₂SO₂CF(CF₃)OCF₃, CF₃CF(OC₃F₇)SO₂CF₂SO₂CF(CF₃)OC₃F₇, C₂F₅SO₂CF₂SO₂C₂F₅, C₂F₅SO₂(CF₂)₂SO₂C₂F₅, C₂F₅SO₂(CF₂)₃SO₂C₂F₅, C₂F₅SO₂(CF₂)₄SO₂C₂F₅, C₃F₇OCF(CF₃)CF₂OCF(CF₃)SO₂CF₂SO₂CF(CF₃)OCF(CF₃)OC₃F₇, (CF₃)₂CFSO₂C₂F₄SO₂CF(CF₃)₂, CF₃CF(OCF₃)SO₂C₂F₄SO₂CF(CF₃)OCF₃CF₃CF(OC₃F₇)SO₂C₂F₄SO₂CF(CF₃)OC₃F₇, C₃F₇OCF(CF₃)C₂F₄OCF(CF₃)SO₂C₂F₄SO₂CF(CF₃)OCF(CF₃)OC₃F₇, (CF₃)₂CFSO₂C₄F₈SO₂CF(CF₃)₂, CF₃CF(OCF₃)SO₂C₄F₈SO₂CF(CF₃)OCF₃CF₃CF(OC₃F₇)SO₂C₄F₈SO₂CF(CF₃)OC₃F₇, C₃F₇OCF(CF₃)C₄F₈OCF(CF₃)SO₂C₄F₈SO₂CF(CF₃)OCF(CF₃)OC₃F₇,

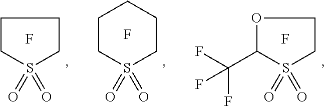

HCF₂CF₂CF₂OCF(CF₃)SO₂CF(CF₃)OCF₂CF₂CF₂H, CH₃OCF₂CF₂CF₂OCF(CF₃)SO₂CF(CF₃)OCF₂CF₂CF₂OCH₃, and CF₃CFHCF₂CF₂OCF(CF₃)SO₂CF(CF₃)OCF₂CF₂CFHCF₃, wherein all appearances of formulas of the type CₙF₂ₙ₊₁ signify any or all isomers of that formula.

Processes for the synthesis of fluorosulfones are well known in the art and are described, for example, in U.S. Pat. No. 6,580,006 and GB 1,189,561, incorporated herein by reference in their entirety, and in S. Temple, J. Org Chem., 1968, 33, 344-346 and R. Lagow, J C S Perkin I, 1979, 2675. Additional processes for synthesizing fluorosulfones are disclosed in the present Examples.

In some embodiments, the present disclosure is further directed to working fluids that include the above-described fluorosulfones as a major component. For example, the working fluids may include at least 25%, at least 50%, at least 70%, at least 80%, at least 90%, at least 95%, or at least 99% by weight of the above-described fluorosulfones, based on the total weight of the working fluid. In addition to the fluorosulfones, the working fluids may include a total of up to 75%, up to 50%, up to 30%, up to 20%, up to 10%, or up to 5% by weight of one or more of the following components: alcohols, ethers, alkanes, alkenes, haloalkenes, perfluorocarbons, perfluorinated tertiary amines, perfluoroethers, cycloalkanes, esters, ketones, oxiranes, aromatics, siloxanes, hydrochlorocarbons, hydrochlorofluorocarbons, hydrofluorocarbons, hydrofluoroolefins, hydrochloroolefins, hydrochlorofluoroolefins, saturated and unsaturated hydrofluoroethers, hydrofluoroketones, hydrofluoronitriles, perfluoroketones, perfluoronitriles, or mixtures thereof, based on the total weight of the working fluid. Such additional components can be chosen to modify or enhance the properties of a composition for a particular use.

It has been discovered that fluorosulfones of the present disclosure have much lower GWP than other highly fluorinated materials known in the art, such as SF₆, HFCs, PFAs, PFPEs, and PFCs. It has been further discovered that, surprisingly, even perfluorosulfones of the present disclosure, despite their completely fluorinated carbon backbones, have much shorter atmospheric lifetimes and correspondingly lower GWPs than other perfluorinated materials, including but not limited to SF₆, PFAs, PFPEs, and PFCs. In some embodiments, the GWP of perfluorosulfones of the present disclosure are more than a factor of 5-10 lower than some of the other perfluorinated materials listed above. That is to say, perfluorosulfones of the present disclosure may have a global warming potential (GWP, 100 year ITH) of less than 2000, or less than 1000, or less than 800, or less than 600.

As used herein, GWP is a relative measure of the global warming potential of a compound based on the structure of the compound. The GWP of a compound, as defined by the Intergovernmental Panel on Climate Change (IPCC) in 1990 and updated in subsequent reports, is calculated as the warming due to the release of 1 kilogram of a compound relative to the warming due to the release of 1 kilogram of CO₂ over a specified integration time horizon (ITH).

$${GWP}_{x} ( {t}^{′} ) = \frac{{\int }_{0}^{ITH} {F}_{x} {C}_{o x} {e}^{- t / {τ}_{x}} d t}{{\int }_{0}^{ITH} {F}_{{CO}_{2}} {C}_{{CO}_{2}} ( t ) d t}$$

where F is the radiative forcing per unit mass of a compound (the change in the flux of radiation through the atmosphere due to the IR absorbance of that compound), Cₒ is the atmospheric concentration of a compound at initial time, □□ is the atmospheric lifetime of a compound, t is time, and x is the compound of interest.

The commonly accepted ITH is 100 years representing a compromise between short-term effects (20 years) and longer-term effects (500 years or longer). The concentration of an organic compound, x, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

In this regard, in some embodiments, the fluorosulfones, or fluorosulfone-containing working or heat transfer fluids of the present disclosure may have a global warming potential (GWP) of less than 2000, 1000, 800, 600, 500, 300, 200, 100 or less than 10.

### Foam Blowing

In some embodiments, the present disclosure relates to the use of the fluorosulfones of the present disclosure as nucleating agents (or foam additives) in the production of polymeric foams and in particular in the production of polyurethane foams or phenolic foams. In this regard, in some embodiments, the present disclosure is directed to a foamable composition that includes one or more blowing agents, one or more foamable polymers or precursor compositions thereof, and one or more nucleating agents that include a fluorosulfone of the present disclosure.

In some embodiments, a variety of blowing agents may be used in the provided foamable compositions including liquid or gaseous blowing agents that are vaporized to foam the polymer or gaseous blowing agents that are generated in situ in order to foam the polymer. Illustrative examples of blowing agents include hydrochlorofluorocarbons (HCFCs), hydrofluorocarbons (HFCs), hydrochlorocarbons (HCCs), iodofluorocarbons (IFCs), hydrocarbons, hydrofluoroolefins (HFOs) and hydrofluoroethers (HFEs). The blowing agent for use in the provided foamable compositions can have a boiling point of from about −45° C. to about 100° C. at atmospheric pressure. Typically, at atmospheric pressure the blowing agent has a boiling point of at least about 15° C., more typically between about 20° C. and about 80° C. The blowing agent can have a boiling point of between about 30° C. and about 65° C. Further illustrative examples of blowing agents that can be used include aliphatic and cycloaliphatic hydrocarbons having about 5 to about 7 carbon atoms, such as n-pentane and cyclopentane, esters such as methyl formate, HFCs such as CF₃CF₂CHFCHFCF₃, CF₃CH₂CF₂H, CF₃CH₂CF₂CH₃, CF₃CF₂H, CH₃CF₂H (HFC-152a), CF₃CH₂CH₂CF₃ and CHF₂CF₂CH₂F, HCFCs such as CH₃CC₁₂F, CF₃CHC₁₂, and CF₂HC₁, HCCs such as 2-chloropropane, and IFCs such as CF₃I, and HFEs such as C₄F₉OCH₃ and HFOs such as CF₃CF═CH₂, CF₃CH═CHF, CF₃CH═CHCl, CF₃CF═CHCl and CF₃CH═CHCF₃ In certain formulations CO₂ generated from the reaction of water with a foam precursor such as an isocyanate can be used as a blowing agent.

In various embodiments, the provided foamable composition may also include one or more foamable polymers or a precursor composition thereof. Foamable polymers suitable for use in the provided foamable compositions include, for example, polyolefins, e.g., polystyrene, poly(vinyl chloride), and polyethylene. Foams can be prepared from styrene polymers using conventional extrusion methods. The blowing agent composition can be injected into a heat-plastified styrene polymer stream within an extruder and admixed therewith prior to extrusion to form a foam. Representative examples of suitable styrene polymers include, for example, the solid homopolymers of styrene, α-methylstyrene, ring-alkylated styrenes, and ring-halogenated styrenes, as well as copolymers of these monomers with minor amounts of other readily copolymerizable olefinic monomers, e.g., methyl methacrylate, acrylonitrile, maleic anhydride, citraconic anhydride, itaconic anhydride, acrylic acid, N-vinylcarbazole, butadiene, and divinylbenzene. Suitable vinyl chloride polymers include, for example, vinyl chloride homopolymer and copolymers of vinyl chloride with other vinyl monomers. Ethylene homopolymers and copolymers of ethylene with, e.g., 2-butene, acrylic acid, propylene, or butadiene may also be useful. Mixtures of different types of polymers can be employed.

In various embodiments, the foamable compositions of the present disclosure may have a molar ratio of nucleating agent to blowing agent of no more than 1:50, 1:25, 1:9, or 1:7, 1:3, or 1:2.

Other conventional components of foam formulations can, optionally, be present in the foamable compositions of the present disclosure. For example, cross-linking or chain-extending agents, foam-stabilizing agents or surfactants, catalysts and fire-retardants can be utilized. Other possible components include fillers (e.g., carbon black), colorants, fungicides, bactericides, antioxidants, reinforcing agents, antistatic agents, plasticizers, and other additives or processing aids.

In some embodiments, polymeric foams can be prepared by vaporizing at least one liquid or gaseous blowing agent or generating at least one gaseous blowing agent in the presence of at least one foamable polymer or a precursor composition thereof and a fluorosulfone nucleating agent as described above. In further embodiments, polymeric foams can be prepared using the provided foamable compositions by vaporizing (e.g., by utilizing the heat of precursor reaction) at least one blowing agent in the presence of a fluorosulfone nucleating agent as described above, at least one organic polyisocyanate and at least one compound containing at least two reactive hydrogen atoms (such as a polyol containing at least two reactive alcohol OH groups). In making a polyisocyanate-based foam, the polyisocyanate, reactive hydrogen-containing compound, nucleating agent, and blowing agent composition can generally be combined, thoroughly mixed (using, e.g., any of the various known types of mixing head and spray apparatus), and permitted to expand and cure into a cellular polymer (closed cell foam). It is often convenient, but not necessary, to pre-blend certain of the components of the foamable composition prior to reaction of the polyisocyanate and the reactive hydrogen-containing compound. For example, it is often useful to first blend the reactive hydrogen-containing compound, blowing agent composition, nucleating agent, and any other components (e.g., surfactant) except the polyisocyanate, and to then combine the resulting mixture with the polyisocyanate. Alternatively, all components of the foamable composition can be introduced separately. It is also possible to pre-react all or a portion of the reactive hydrogen-containing compound with the polyisocyanate to form a prepolymer.

### Dielectric/Insulating Gas

It is common in electrical power generation and transmission systems to use dielectric gases to insulate switches, circuit breakers, transmission lines, and other equipment operating at very high voltages and high current densities. SF₆ is a strongly electronegative gas with a high dielectric strength. Its breakdown voltage is nearly three times that of air under ambient conditions. It also has good heat transfer properties and partially reforms itself when dissociated under the high temperature conditions of an electrical discharge thus retaining its insulating properties over time. Most of the stable decomposition products of SF₆ do not degrade its insulating properties. It does not produce polymerization products or conductive particles or deposits during arcing. SF₆ is chemically compatible with materials of construction (insulating and conductive) in various electrical equipment such as transformers, switch gears, etc. These properties have made SF₆ the dielectric gas of choice for the electric power industry for many years.

However, SF₆ can form highly toxic products such as S₂F₁₀ and SO₂F₂ as a result of electrical discharges. Precautions are necessary to avoid contact with spent dielectric gas as a result. SF₆ is also the most potent greenhouse gas known, with a GWP 22,200 times that of CO₂. It has an atmospheric lifetime of 3200 years because of its very high chemical stability. Potential substitutes include PFCs, nitrogen, and carbon dioxide. Many PFCs are better dielectrics than SF₆ due in part to their higher molecular weights, but are prone to producing conducting carbon particles that degrade performance over time. Dilutions of PFCs with nitrogen reduce this tendency. However, PFCs are also potent greenhouse gases.

Dry nitrogen and carbon dioxide are slightly better dielectrics than air principally due to the removal of water vapor. They have been examined for their potential to replace SF₆, but they are not sufficiently insulating in all applications and equipment.

In accordance with the present disclosure, certain fluorosulfones have been found to provide the desirable performance properties of SF₆, including high dielectric strength, good heat transfer properties, and stability. In addition, fluorosulfones are much more readily degraded in the atmosphere. This reduces their atmospheric lifetimes and thus their contribution as a greenhouse gas is low and much more acceptable than SF₆ or PFCs, for example. In this regard, in some embodiments, the present disclosure is directed to dielectric fluids that include one or more fluorosulfones of the present disclosure, as well as to electrical devices (e.g., capacitors, switchgear, transformers, or electric cables or buses) that include such dielectric fluids. For purposes of the present application, the term “dielectric fluid” is inclusive of both liquid dielectrics and gaseous dielectrics. The physical state of the fluid, gaseous or liquid, is determined by the operating conditions of temperature and pressure of the electrical device in which it is used and the thermophysical properties of the fluid or fluid mixture. In some embodiments, the present disclosure is directed to dielectric gases that include one or more fluorosulfones of the present disclosure, as well as to electrical devices (e.g., capacitors, switchgear, transformers, or electric cables or buses) that include such dielectric gases.

In some embodiments, the dielectric fluids include one or more fluorosulfones of the present disclosure (e.g., one or more gaseous fluorosulfones) and, optionally, one or more other dielectric fluids. The other dielectric fluid may be a non-condensable gas or an inert gas or another highly fluorinated dielectric gas. Suitable other dielectric fluids include, but are not limited to, air, nitrogen, nitrous oxide, oxygen, helium, argon, carbon dioxide, heptafluoroisobutyronitrile, 1,1,1,3,4,4,4-heptafluoro-3-(trifluoromethyl)butan-2-one, SF₆, and 2,3,3,3-tetrafluoro-2-(trifluoromethoxy)propanenitrile or combinations thereof, for example. Generally, the other dielectric fluid may be used in amounts such that vapor pressure is at least 70 kPa at 25° C., or at the operating temperature of the electrical device.

In some embodiments, the fluorosulfone containing dielectric fluids of the present disclosure may include fluorosulfones alone or in mixtures with one, two, three or even four or more other dielectric fluids including, but not limited to, heptafluoroisobutyronitrile, 1,1,1,3,4,4,4-heptafluoro-3-(trifluoromethyl)butan-2-one, 2,3,3,3-tetrafluoro-2-(trifluoromethoxy)propanenitrile, SF₆, nitrogen, carbon dioxide, nitrous oxide, oxygen, air, helium, or argon. In the context of the present disclosure, oxygen, when used as a dielectric dilution gas, is used in “small quantity”, meaning that the oxygen is present in the overall gas mixture at a mole percentage in the range of 1-25% or 2-15% or 2-10%.

In some embodiments, the fluorosulfone component of the dielectric fluid of the present disclosure is perfluorinated.

In other embodiments, the fluorosulfone dielectric fluids and other dielectric fluids are dry, meaning the water content of the fluid is less than 500 ppm, less than 300 ppm, less than 100 ppm, less than 50 ppm, less than 30 ppm, or less than 10 ppm by weight.

Illustrative examples of fluorosulfones suitable for use in such applications include, but are not limited to, bis(trifluoromethyl)sulfone, trifluoromethylpentafluoroethylsulfone, perfluorodiethylsulfone, or mixtures of one or more fluorosulfones of the present disclosure with a significant vapor pressure (in some embodiments greater than or equal to about 0.05 atm, greater than or equal to about 0.1 atm, greater than or equal to about 0.2 atm, greater than or equal to about 0.3 atm, or even greater than or equal to about 0.4 atm) over the temperature range of about −20° C. to about 50° C.

The dielectric fluids of the present application may be useful for electrical insulation and for arc quenching and current interruption equipment used in the transmission and distribution of electrical energy. Generally, there are three major types of electrical devices in which the fluids of the present disclosure can be used: (1) gas-insulated circuit breakers and current-interruption equipment, (2) gas-insulated transmission lines, and (3) gas-insulated transformers. Such gas-insulated equipment is a major component of power transmission and distribution systems.

The above described dielectric fluids and fluid mixtures of this disclosure provide significant advantages and benefits when used in medium and high voltage electrical equipment. These include, but are not restricted to, high dielectric strength, non-flammability, low toxicity, low global warming potential, good heat transfer properties, and good stability in the application.

In some embodiments, the present disclosure provides electrical devices, such as capacitors, comprising metal electrodes spaced from each other such that the gaseous dielectric fills the space between the electrodes. The interior space of the electrical device may also comprise a reservoir of the liquid dielectric fluid which is in equilibrium with the gaseous dielectric fluid. Thus, the reservoir may replenish any losses of the dielectric fluid.

### Organic Rankine Cycle

The rising cost of energy, mounting concern over emissions of greenhouse gases, and limitations of the power grid have spawned interest in renewable energy sources, localized or regional power generation, and technologies that make use of energy that would otherwise be wasted. Among the latter is Organic Rankine Cycle (ORC) technology. ORC is similar to the conventional steam Rankine cycle used in power plants except that the ORC plant is generally sized below 10 megaWatts and usually operates at much lower temperatures, at which steam from water is no longer an ideal working fluid and lower boiling organic fluids such as hydrocarbon pentane are preferred. Hydrocarbons are environmentally quite benign, but due to flammability are often considered too dangerous for use in ORCs, particularly close-coupled ones installed to capture energy from, for example, cement drying plants, internal combustion engine exhaust manifolds, etc.

Nonflammable working fluids are preferred, but the list of suitable candidates is short. Chlorofluorocarbons (CFCs), HCFCs, and brominated materials are excluded as they are ozone depleting. Perfluorocarbon (PFC) fluids have long been suggested as candidates. HFCs more recently have been examined in these applications. However, both PFCs and HFCs are designated for reduced emissions due to their high GWPs and have fallen out of favor particularly in the European Union and Japan. HFEs have suitable performance properties, but may lack sufficient thermal stability to be used in some ORC applications. Fluoroketones have been suggested as viable candidates, but may also not be sufficiently stable for long term use in an ORC.

Fluorosulfones of the present disclosure generally have the physical and thermal properties needed to be suitable as ORC working fluids and are projected to be sufficiently stable for the application, while also providing relatively low GWPs compared to PFCs, PFAs, PFPEs and HFCs. This combination of properties make them good candidates for ORC working fluids. In some embodiments the fluorosulfones are perfluorinated.

In some embodiments, the present disclosure is directed to an apparatus for converting thermal energy into mechanical energy in a Rankine cycle (e.g., an ORC). The apparatus may include a working fluid that includes one or more fluorosulfones of the present disclosure. The apparatus may further include a heat source to vaporize the working fluid and form a vaporized working fluid, a turbine through which the vaporized working fluid is passed thereby converting thermal energy into mechanical energy, a condenser to cool the vaporized working fluid after it is passed through the turbine, and a pump to recirculate the working fluid.

In some embodiments, the present disclosure relates to a process for converting thermal energy into mechanical energy in a Rankine cycle. The process may include using a heat source to vaporize a working fluid that includes one or more fluorosulfones of the present disclosure to form a vaporized working fluid. In some embodiments, the heat is transferred from the heat source to the working fluid in an evaporator or boiler. The vaporized working fluid may be pressurized and can be used to do work by expansion. The heat source can be of any form such as from fossil fuels, e.g., oil, coal, or natural gas. Additionally, in some embodiments, the heat source can come from nuclear power, solar power, or fuel cells. In other embodiments, the heat can be “waste heat” from other heat transfer systems that would otherwise be lost to the atmosphere. The “waste heat,” in some embodiments, can be heat that is recovered from a second Rankine cycle system from the condenser or other cooling device in the second Rankine cycle.

An additional source of “waste heat” can be found at landfills where methane gas is flared off. In order to prevent methane gas from entering the environment and thus contributing to global warming, the methane gas generated by the landfills can be burned by way of “flares” producing carbon dioxide and water which are both less harmful to the environment in terms of global warming potential than methane. Other sources of “waste heat” that can be useful in the provided processes are geothermal sources and heat from other types of engines such as gas turbine engines that give off significant heat in their exhaust gases and to cooling liquids such as water and lubricants.

In the provided processes, the vaporized working fluid may be expanded though a device that can convert the pressurized working fluid into mechanical energy. In some embodiments, the vaporized working fluid is expanded through a turbine which can cause a shaft to rotate from the pressure of the vaporized working fluid expanding. The turbine can then be used to do mechanical work such as, in some embodiments, operate a generator, thus generating electricity. In other embodiments, the turbine can be used to drive belts, wheels, gears, or other devices that can transfer mechanical work or energy for use in attached or linked devices.

After the vaporized working fluid has been converted to mechanical energy the vaporized (and now expanded) working fluid can be condensed using a cooling source to liquefy for reuse. The heat released by the condenser can be used for other purposes including being recycled into the same or another Rankine cycle system, thus saving energy. Finally, the condensed working fluid can be pumped by way of a pump back into the boiler or evaporator for reuse in a closed system.

The desired thermodynamic characteristics of organic Rankine cycle working fluids are well known to those of ordinary skill and are discussed, for example, in U.S. Pat. Appl. Publ. No. 2010/0139274 (Zyhowski et al.). The greater the difference between the temperature of the heat source and the temperature of the condensed liquid or a provided heat sink after condensation, the higher the Rankine cycle thermodynamic efficiency. The thermodynamic efficiency is influenced by matching the working fluid to the heat source temperature. The closer the evaporating temperature of the working fluid to the source temperature, the higher the efficiency of the system. Toluene can be used, for example, in the temperature range of 79° C. to about 260° C., however toluene has toxicological and flammability concerns. Fluids such as 1,1-dichloro-2,2,2-trifluoroethane and 1,1,1,3,3-pentafluoropropane can be used in this temperature range as an alternative. But 1,1-dichloro-2,2,2-trifluoroethane can form toxic compounds below 300° C. and needs to be limited to an evaporating temperature of about 93° C. to about 121° C. Thus, there is a desire for other environmentally-friendly Rankine cycle working fluids with higher critical temperatures so that source temperatures such as gas turbine and internal combustion engine exhaust can be better matched to the working fluid.

In some embodiments, the fluorosulfones of the present disclosure useful for Rankine cycle working fluids may have boiling points from about 10° C. to about 120° C. (in some embodiments about 10° C. to about 20° C., about 20° C. to about 50° C., about 50° C. to 80° C. or even about 80° C. to about 120° C.) alone or in combination with other fluorosulfones or other fluids as the working fluid.

### Direct Contact Electronic Immersion Cooling

For decades PFC fluids have been used in specialty, usually high value electronic cooling applications and were often placed in direct contact with the electronics being cooled. Examples include military electronics and supercomputer applications. PFC fluids were favored because they are very inert and excellent dielectrics. More recently HFCs, HFEs, and PFKs have been examined for these applications.

More mainstream electronics like servers and desktop computers have historically used air cooling, but recently the demand for more computing power has caused chip powers to rise to the level that liquid cooling has begun to emerge in high performance machines, due to improved efficiency. Aqueous working fluids are preferred from a performance standpoint in indirect contact liquid phase systems, but raise reliability concerns due to their propensity to cause short circuits if a leak should develop. Dielectric liquids should be nonflammable for similar reasons, since a fire could break out in the event of a leak. A dielectric liquid's environmental properties must also be consistent with the environmental requirements of the computer manufacturer and its customers. PFC liquids (including perfluorinated hydrocarbon, perfluorinated amine and perfluorinated ether and polyether liquids) and HFC liquids are not ideal candidates for this application due to their high GWPs, thus there is a continuing need to develop materials that can provide improved environmental profiles, while also satisfying all the other requirements for direct contact electronic immersion cooling.

Fluorosulfones of the present disclosure generally meet the performance and environmental requirements for this application. Their safety, nonflammability, high dielectric strength, low volume resistivity, material compatibility, and excellent heat transfer properties are suitable for direct contact cooling and use with highly valuable electronics with excellent reliability. In addition, their short atmospheric lifetime translates to significantly reduced GWP and minimal impact as greenhouse gases.

For example, modern power semiconductors like Field Effect Transistors (FETs) and Insulated Gate Bipolar Transistors (IGBTs) generate very high heat fluxes. These devices are used in the power converter modules in hybrid electric vehicles. These devices must function under conditions of extreme heat and cold and this has spurred the adoption of direct contact cooling technologies. The liquids used in these applications must again be electrically insulating, non-flammable, compatible with the electronic components they are in contact with, and provide a level of environmental sustainability consistent with the environmental goals of the hybrid technology. Fluorosulfones of the present disclosure generally meet these requirements.

The fluorosulfones of the present disclosure, alone or in combination, may be employed as fluids for transferring heat from various electronic components by direct contact to provide thermal management and maintain optimal component performance under extreme operation conditions. Illustrative materials are fluorosulfones with boiling points from about 10° C. to about 150° C. (in some embodiments from about about 10° C. to about 25° C., about 25° C. to about 50° C., or even about 50° C. to about 150° C.). In some embodiments, the fluorosulfones are perfluorinated.

Direct contact fluid immersion technology is well known to be useful for thermal management of electronic components. Hydrofluoroethers and perfluoroketones are two examples of environmentally sustainable chemistries that have been used for many years in direct contact fluid immersion heat transfer applications that place stringent performance requirements on the fluids employed, such as non-flammability, low toxicity, small environmental footprint (zero ODP, low GWP), high dielectric strength, low dielectric constant, high volume resistivity, stability, and good thermal properties. These fluids have found use in many thermal management applications that include semiconductor manufacturing, and electronics cooling (e.g. power electronics, transformers and computers/servers). Surprisingly, it has been discovered that perfluorinated sulfones of the present disclosure generally provide improved dielectric properties compared to hydrofluoroethers, including lower dielectric constant, higher dielectric strength, and higher volume resistivity. The perfluorinated sulfones also provide higher heats of vaporization than the HFEs or the perfluoroketones and excellent heat transfer coefficients for improved heat transfer performance in two-phase immersion applications. Furthermore, it has been discovered that fluorosulfones generally provide improved hydrolytic stability compared to perfluoroketones and HFEs. Thus, fluorosulfones of the present disclosure have recently been found to provide a unique balance of properties that makes them highly attractive fluid candidates for use in direct contact immersion cooling applications.

In some embodiments, the present disclosure describes the use of fluorosulfones as two-phase immersion cooling fluids for electronic devices, including computer servers.

Large scale computer server systems can perform significant workloads and generate a large amount of heat during their operation. A significant portion of the heat is generated by the operation of these servers. Due in part to the large amount of heat generated, these servers are typically rack mounted and air-cooled via internal fans and/or fans attached to the back of the rack or elsewhere within the server ecosystem. As the need for access to greater and greater processing and storage resources continues to expand, the density of server systems (i.e., the amount of processing power and/or storage placed on a single server, the number of servers placed in a single rack, and/or the number of servers and or racks deployed on a single server farm), continue to increase. With the desire for increasing processing or storage density in these server systems, the thermal challenges that result remain a significant obstacle. Conventional air cooling systems (e.g., fan based) require large amounts of power, and the cost of power required to drive such systems increases exponentially with the increase in server densities. Consequently, there exists a need for an efficient, low power usage system for cooling the servers, while allowing for the desired increased processing and/or storage densities of modern server systems.

Two-phase immersion cooling is an emerging cooling technology for the high-performance server computing market which relies on the heat absorbed in the process of vaporizing a liquid (the cooling fluid) to a gas (i.e., the heat of vaporization). The fluids used in this application must meet certain requirements to be viable in the application. For example, the boiling temperature during operation should be in a range between for example 45° C.-75° C. Generally, this range accommodates maintaining the server components at a sufficiently cool temperature while allowing heat to be dissipated efficiently to an ultimate heat sink (e.g., outside air). The fluid must be inert so that it is compatible with the materials of construction and the electrical components. The fluid should be stable such that it does not react with common contaminants such as water or with reagents such as activated carbon or alumina that might be used to scrub the fluid during operation. The global warming potential (GWP, 100 yr ITH) and ozone depletion potential (ODP) of the parent compound and its degradation products should be below acceptable limits, for example, a GWP less than 2000, 1000, 800 or 600 and an ODP less than 0.01, respectively. Fluorosulfones of the present disclosure generally meet these requirements.

In another embodiment, the present invention describes the use of fluorosulfones as single-phase immersion cooling fluids for electronics. Single phase immersion cooling has a long history in computer server cooling. There is no phase change in single phase immersion. Instead the liquid warms and cools as it flows or is pumped through the computer hardware and a heat exchanger, respectively, thereby transferring heat away from the server. The fluids used in single phase immersion cooling of servers must meet the same requirements as outlined above except that they typically have higher boiling temperatures exceeding about 75 degrees C. to limit evaporative losses. Fluorosulfones of the present disclosure generally meet these requirements.

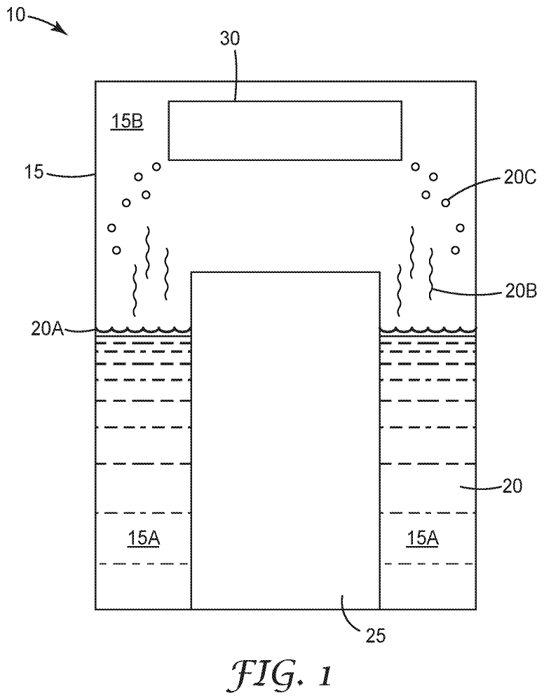

In some embodiments, the present disclosure may be directed to an immersion cooling system that includes the above-discussed fluorosulfone-containing working fluids. Generally, the immersion cooling systems may operate as two-phase vaporization-condensation cooling vessels for cooling one or more heat generating components. As shown in FIG. 1, in some embodiments, a two-phase immersion cooling system 10 may include a housing 10 having an interior space 15. Within a lower volume 15A of interior space 15, a liquid phase 20 of a fluorosulfone-containing working fluid having an upper liquid surface 20A (i.e., the topmost level of the liquid phase 20) may be disposed. The interior space 15 may also include an upper volume 15B extending from the liquid surface 20A up to an upper portion 10A of the housing 10.

In some embodiments, a heat generating component 25 may be disposed within the interior space 15 such that it is at least partially immersed (and up to fully immersed) in the liquid phase 20 of the working fluid. That is, while heat generating component 25 is illustrated as being only partially submerged below the upper liquid surface 20A, in some embodiments, the heat generating component 25 may be fully submerged below the liquid surface 20A. In some embodiments, the heat generating components may include one or more electronic devices, such as computer servers.

In various embodiments, a heat exchanger 30 (e.g., a condenser) may be disposed within the upper volume 15B. Generally, the heat exchanger 30 may be configured such that it is able to condense a vapor phase 20B of the working fluid that is generated as a result of the heat that is produced by the heat generating element 25. For example, the heat exchanger 30 may have an external surface that is maintained at a temperature that is lower than the condensation temperature of a vapor phase of the working fluid. In this regard, at the heat exchanger 30, a rising vapor phase 20B of the working fluid may be condensed back to liquid phase or condensate 20C by releasing latent heat to the heat exchanger 30 as the rising vapor phase 20B comes into contact with the heat exchanger 30. The resulting condensate 20C may then be returned to the liquid phase 20 disposed in the lower volume of 15 A.

In some embodiments, the present disclosure may be directed to an immersion cooling system which operates by single-phase immersion cooling. Generally, the single phase immersion cooling system is similar to that of the two-phase system in that it may include a heat generating component disposed within the interior space of a housing such that it is at least partially immersed (and up to fully immersed) in the liquid phase of the 15 working fluid. The single-phase system may further include a pump and a heat exchanger,

the pump operating to move the working fluid to and from the heat generating components and the heat exchanger, and the heat exchanger operating to cool the working fluid. The heat exchanger may be disposed within or external to the housing.

While the present disclosure depicts a particular example of a suitable two-phase immersion cooling system in FIG. 1, it is to be appreciated that the benefits and advantages of the fluorosulfone-containing working fluids of the present disclosure may be realized in any known two-phase or single-phase immersion cooling system.

In some embodiments, the present disclosure may be directed to methods for cooling electronic components. Generally, the methods may include at least partially immersing a heat electronic generating component (e.g., a computer server) in a liquid that includes the above-described fluorosulfones or working fluid. The method may further include transferring heat from the heat generating electronic component using the above-described fluorosulfone or working fluid.

### Direct Contact Immersion Battery Thermal Management

Electrochemical cells (e.g., lithium-ion batteries) are in widespread use worldwide in a vast array of electronic and electric devices ranging from hybrid and electric vehicles to power tools, portable computers, and mobile devices. While generally safe and reliable energy storage devices, lithium-ion batteries are subject to catastrophic failure known as thermal runaway under certain conditions. Thermal runaway is a series of internal exothermic reactions that are triggered by heat. The creation of excessive heat can be from electrical over-charge, thermal over-heat, or from an internal electrical short. Internal shorts are typically caused by manufacturing defects or impurities, dendritic lithium formation and mechanical damage. While there is typically protective circuitry in the charging devices and in the battery packs that will disable the battery in the event of overcharging or overheating, it cannot protect the battery from internal shorts caused by internal defects or mechanical damage.

A thermal management system for lithium-ion battery packs is often required to maximize the cycle life of lithium-ion batteries. This type of system maintains uniform temperatures of each cell within a battery pack. High temperatures can increase the capacity fade rate and impedance of lithium-ion batteries while decreasing their lifespan. Ideally, each individual cell within a battery pack will be at the same ambient temperature.

Direct contact fluid immersion of batteries can mitigate low probability, but catastrophic, thermal runaway events while also providing necessary ongoing thermal management for the efficient normal operation of the lithium-ion battery packs. This type of application provides thermal management when the fluid is used with a heat exchange system to maintain a desirable operational temperature range. However, in the event of mechanical damage or an internal short of any of the lithium-ion cells, the fluid would also prevent propagation or cascading of the thermal runaway event to adjacent cells in the pack via evaporative cooling, thus significantly mitigating the risk of a catastrophic thermal runaway event involving multiple cells. As with immersion cooling of electronics described above, immersion cooling and thermal management of batteries can be achieved using a system designed for single phase or two-phase immersion cooling and the fluid requirements for battery cooling are similar to those described above for electronics. In either scenario, the fluids are disposed in thermal communication with the batteries to maintain, increase, or decrease the temperature of the batteries (i.e., heat may be transferred to or from the batteries via the fluid).

Direct contact fluid immersion technology has been shown to be useful for thermal management of batteries and for providing thermal runaway protection, but there is still a need for improved fluids that can provide better chemical stability and system longevity. Hydrofluoroethers and perfluoroketones are two examples of chemistries that have shown utility in direct contact fluid immersion heat transfer applications for thermal management and thermal runaway protection of batteries, while also providing acceptable global warming potentials. These applications place stringent performance requirements on the fluids employed, such as non-flammability, low toxicity, small environmental footprint, high dielectric strength, low dielectric constant, high volume resistivity, stability, materials compatibility, and good thermal properties. Surprisingly, it has been discovered that fluorosulfones, and particularly perfluorosulfones, of the present disclosure generally provide improved dielectric properties compared to saturated and unsaturated hydrofluoroethers, including lower dielectric constant, higher dielectric strength, and higher volume resistivity. Low dielectric constants can be important for keeping levels of dissolved ionic impurities at low levels in the fluid to maintain high volume resistivity over long periods. These ionic impurities can originate from the materials of construction of the battery pack or from the individual cells (from electrolyte leakage) and can get extracted into the heat transfer fluid over time, thereby adversely altering the fluid properties. High dielectric strength is important in preventing arcing at high voltages. Fluorosulfones of the present disclosure also provide higher heats of vaporization than hydrofluoroethers, perfluoroketones, or perfluorinated fluids, such as PFCs, PFAs or PFPEs, for improved heat transfer performance in two-phase immersion applications. Furthermore, it has been discovered that fluorosulfones of the present disclosure provide improved hydrolytic stability compared to perfluoroketones and HFEs. Hydrolytic degradation of fluids can produce ionic contaminants that can cause corrosion or compromise battery performance. Thus, fluorosulfones of the present disclosure have been found to provide a unique balance of properties that makes them highly attractive fluid candidates for use in direct contact immersion cooling and thermal management applications for batteries, while also providing low global warming potentials. Consequently, in some embodiments, the present disclosure is directed to a thermal management system for a lithium-ion battery pack. The system may include a lithium-ion battery pack and a working fluid in thermal communication with the lithium-ion battery pack. The working fluid may include one or more of the fluorosulfones of the present disclosure (e.g., perfluorosulfones).

### High Temperature Heat Exchange

In some embodiments, the fluorosulfones of the present disclosure (or working or heat transfer fluids containing the same) can be used in various applications as heat transfer agents (for example, for the cooling or heating of integrated circuit tools in the semiconductor industry, including tools such as dry etchers, integrated circuit testers, photolithography exposure tools (steppers), ashers, chemical vapor deposition equipment, automated test equipment (probers), physical vapor deposition equipment (e.g. sputterers), and vapor phase soldering fluids, and thermal shock fluids).

In some embodiments, the present disclosure is further directed to an apparatus for heat transfer that includes a device and a mechanism for transferring heat to or from the device. The mechanism for transferring heat may include a heat transfer or working fluid that includes one or more fluorosulfones of the present disclosure.

The provided apparatus for heat transfer may include a device. The device may be a component, work-piece, assembly, etc. to be cooled, heated or maintained at a predetermined temperature or temperature range. Such devices include electrical components, mechanical components and optical components. Examples of devices of the present disclosure include, but are not limited to microprocessors, wafers used to manufacture semiconductor devices, power control semiconductors, electrical distribution switch gear, power transformers, circuit boards, multi-chip modules, packaged and unpackaged semiconductor devices, lasers, chemical reactors, fuel cells, heat exchangers, and electrochemical cells. In some embodiments, the device can include a chiller, a heater, or a combination thereof.

In yet other embodiments, the devices can include electronic devices, such as processors, including microprocessors. As these electronic devices become more powerful, the amount of heat generated per unit time increases. Therefore, the mechanism of heat transfer plays an important role in processor performance. The heat-transfer fluid typically has good heat transfer performance, good electrical compatibility (even if used in “indirect contact” applications such as those employing cold plates), as well as low toxicity, low (or non-) flammability and low environmental impact. Good electrical compatibility requires that the heat-transfer fluid candidate exhibit high dielectric strength, high volume resistivity, and poor solvency for polar materials. Additionally, the heat-transfer fluid should exhibit good mechanical compatibility, that is, it should not affect typical materials of construction in an adverse manner, and it should have a low pour point and low viscosity to maintain fluidity during low temperature operation.

The provided apparatus may include a mechanism for transferring heat. The mechanism may include a heat transfer fluid. The heat transfer fluid may include one or more fluorosulfones of the present disclosure. Heat may be transferred by placing the heat transfer mechanism in thermal contact with the device. The heat transfer mechanism, when placed in thermal contact with the device, removes heat from the device or provides heat to the device, or maintains the device at a selected temperature or temperature range. The direction of heat flow (from device or to device) is determined by the relative temperature difference between the device and the heat transfer mechanism.

The heat transfer mechanism may include facilities for managing the heat-transfer fluid, including, but not limited to pumps, valves, fluid containment systems, pressure control systems, condensers, heat exchangers, heat sources, heat sinks, refrigeration systems, active temperature control systems, and passive temperature control systems. Examples of suitable heat transfer mechanisms include, but are not limited to, temperature controlled wafer chucks in plasma enhanced chemical vapor deposition (PECVD) tools, temperature-controlled test heads for die performance testing, temperature-controlled work zones within semiconductor process equipment, thermal shock test bath liquid reservoirs, and constant temperature baths. In some systems, such as etchers, ashers, PECVD chambers, vapor phase soldering devices, and thermal shock testers, the upper desired operating temperature may be as high as 170° C., as high as 200° C., or even as high as 230° C.

Heat can be transferred by placing the heat transfer mechanism in thermal communication with the device. The heat transfer mechanism, when placed in thermal communication with the device, removes heat from the device or provides heat to the device, or maintains the device at a selected temperature or temperature range. The direction of heat flow (from device or to device) is determined by the relative temperature difference between the device and the heat transfer mechanism. The provided apparatus can also include refrigeration systems, cooling systems, testing equipment and machining equipment. In some embodiments, the provided apparatus can be a constant temperature bath or a thermal shock test bath.

Fluorosulfones of the present disclosure, which exhibit unexpectedly high thermal stabilities, can be particularly useful in high temperature applications. In some embodiments, fluorosulfones of the present disclosure that have boiling points between about 150° C. and about 300° C. (in some embodiments from about 180 to about 290, about 200 to about 280, or even about 220 to about 260° C.) can be used for vapor phase soldering of lead-free solders. Fluorosulfones that have boiling points above about 70° C. (in some embodiments above about 100° C., above about 130° C., or even above about 150° C.), as well as viscosity less than about 30 centiStokes at −40° C. (in some embodiments at about −20° C. and in other embodiments at about 25° C.), are particularly useful in the types of heat transfer applications that require both high temperature and low temperature operation. In some embodiments, the fluorosulfones are perfluorinated.

### Vapor Reactor Cleaning, Etching, and Doping Gases

Chemical vapor deposition chambers, physical vapor deposition chambers, and etching chambers are widely used in the semiconductor industry in connection with the manufacture of various electronic devices and components. Such chambers use reactive gases or vapors to deposit, pattern or remove various dielectric and metallic materials. PFCs such as C₂F₆ are widely used in conjunction with vapor reactors for etching or patterning materials and for removing unwanted deposits that build-up on the reactor walls and parts. When combined with oxygen in a radio frequency plasma, these PFCs provide the ability to generate various radicals such as CF₃. and CF₂: and atomic fluorine useful in the vapor reaction processes. However, these PFCs have long atmospheric lifetimes and high GWPs. As a result, the semiconductor industry is attempting to reduce the emission of these compounds to the environment. The industry has expressed a need for alternative chemicals for vapor reaction techniques that do not contribute to global warming.

In some embodiments, the present disclosure provides methods of using a fluorosulfone in a vapor reactor as a reactive gas to remove unwanted deposits, to etch dielectric and metallic materials, and to dope materials. Fluorosulfones of the present disclosure have shorter atmospheric lifetimes and lower global warming potentials compared to the PFCs traditionally used in this application. Like PFCs, fluorosulfones, such as C₂F₅SO₂C₂F₅ and CF₃SO₂CF₃, provide the ability to generate various radicals such as CF₃. and CF₂: and atomic fluorine in vapor reaction processes. However, fluorosulfones of the present disclosure also offer the advantage of significantly reducing greenhouse gas emissions from these processes due to their lower GWP.

Illustrative examples of fluorosulfones suitable for uses such as vapor reactor cleaning, etching, and doping gases include those with boiling points less than about 150° C. (in some embodiments less than about 130° C., less than about 100° C., or even less than about 80° C.). In some embodiments the fluorosulfones are perfluorinated.

### Protective Cover Agents for Molten Active Metals

Parts made with magnesium (or its alloys) with high strength-to-weight ratios and good electromagnetic shielding properties are finding increasing use as components in the automobile, aerospace, and electronics industries. These components are typically manufactured by casting techniques where the magnesium metal or its alloy is heated to a molten state at temperatures as high as 1400° F. (800° C.) and the resulting liquid metal is poured or pumped into molds or dies to form components or parts. In the case of primary metal production similar casting of molten purified metal or alloyed metal is done to form ingots of various sizes and shapes.

While magnesium is in the molten state it is necessary to protect it from reacting with atmospheric oxygen. This reaction is a spontaneous, exothermic one that is very difficult to extinguish and therefore very destructive to manufacturing equipment and facilities as well as a danger to factory workers and emergency response personnel. A secondary, but equally important purpose for protecting molten magnesium is the prevention of sublimation of magnesium vapors to cooler portions of the casting apparatus. Such sublimed solids are also very susceptible to ignition in the presence of air. Both molten magnesium and sublimed magnesium vapors can produce an extremely hot magnesium fire potentially causing extensive property damage and serious injury or loss of human life. Similarly, other reactive metals such as aluminum, lithium, calcium, strontium, and their alloys are highly reactive in their molten state, necessitating protection from atmospheric air or oxygen.

Various methods have been used to minimize the exposure of molten magnesium or other reactive metals to air. The two most viable methods are the use of salt fluxes and the use of cover gases or protective atmospheres. Salt fluxes are liquid at magnesium melt temperatures and form an impervious layer floating on the molten metal surface that effectively separates the molten metal from air. However, fluxes have the disadvantages of oxidizing at elevated temperatures and forming a thick hardened layer of metal oxides and/or metal chlorides, which can be easily cracked, potentially exposing the molten metal to air. Also, inclusion of liquid flux into the melt can occur when ingots are added to a molten metal bath. Such inclusions produce sites that initiate corrosion of the cast parts and degrade the physical properties of the metal parts produced. Finally, the dust particles and fumes from the use of flux can cause serious corrosion problems to ferrous metals in the foundry and pose a serious safety problem for foundry workers.

As a result, magnesium foundries have shifted to protective cover gases, which form a thin protective film on the surface of the molten magnesium. This protective film effectively separates the reactive metal from oxygen and prevents destructive fires or troublesome metal inclusions of oxides and fluxes. The cover gas agent of choice is SF₆ due to its high degree of stability and low toxicity. SF₆ is so stable that it largely survives exposure to molten magnesium and is emitted to the atmosphere. SF₆'s long atmospheric lifetime coupled with a very high infrared absorption cross-section results in its exceedingly high GWP, i.e., 22,200 times greater than CO₂ (100 year ITH), and a need to replace it.

The requirements for an effective cover gas agent as a substitute for SF₆ are that it be effective in forming a protective surface film on molten magnesium and molten magnesium alloys, have a short atmospheric lifetime and/or have a low infrared absorption cross-section (low GWP), have essentially no ozone depletion potential, be non-flammable and of low toxicity, produce little or no harmful degradation products when exposed to molten magnesium, be readily available, low cost, and be compatible with existing processes and equipment.

Currently, several possible substitutes are being examined which include SO₂, HFCs, e.g., HFC-134a and HFC-125, and fluorinated ketones such as C₂F₅C(O)CF(CF₃)₂. Sulfur dioxide (SO₂) has long been known to protect molten magnesium by forming a MgSO₄-containing film. However, the toxic properties of SO₂ (permissible exposure limit (PEL)=2 ppmV) make it difficult and costly to use safely. The fluorine of HFCs and fluorinated ketones readily forms MgF₂ and becomes part of the surface layer on molten magnesium. The significant GWP of HFCs and possible problems with HF production HFCs also reduce HFC usefulness.

Fluorosulfones of the present disclosure are useful in this application and provide a more environmentally acceptable material. Fluorosulfones in contact with molten magnesium form a protective surface film that provides a reliable and safe protective cover. Like other cover gas agents, fluorosulfones are compatible with a number of carrier gases such as dry air, nitrogen, carbon dioxide, and argon alone or in mixtures. Effective concentrations of fluorosulfones in carrier gas range from about 0.01 to about 5.0 volume percent depending upon the process and alloy that is being protected and/or the specific process parameters (temperature, cover gas flow rates, distribution systems, and equipment) being used.

In some embodiments, the present disclosure provides compositions of cover gases and a method of using cover gases for protection of molten reactive metals comprised of a fluorosulfone of the present disclosure at a concentration of about 0.01 to about 5 volume percent in dry air, nitrogen, carbon dioxide, argon or mixtures of these. The cover gas mixture is distributed over the molten metal producing a protective surface film that prevents the metal from burning. In some embodiments, the fluorosulfones are perfluorinated.

### LISTING OF EMBODIMENTS

1. A foamable composition comprising:

a blowing agent;

a foamable polymer or a precursor composition thereof; and

a nucleating agent, wherein said nucleating agent comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

2. A foamable composition according to embodiment 1, wherein R¹, R², and R³ are perfluorinated.
3. A foamable composition according to any one of embodiments 1-2, wherein the nucleating agent and the blowing agent are in a molar ratio of less than 1:2.
4. A foamable composition according to any one of embodiments 1-3, wherein the blowing agent comprises an aliphatic hydrocarbon having from about 5 to about 7 carbon atoms, a cycloaliphatic hydrocarbon having from about 5 to about 7 carbon atoms, a hydrocarbon ester, water, or combinations thereof.
5. A foamable composition according to any one of embodiments 1-4, wherein the compound of structural formula (I) has a GWP (100 year ITH) of less than 2000.
6. A foam made with the foamable composition according to any one of embodiments 1-5.
7. A process for preparing polymeric foam comprising:

vaporizing at least one liquid or gaseous blowing agent or generating at least one gaseous blowing agent in the presence of at least one foamable polymer or a precursor composition thereof and a nucleating agent, wherein said nucleating agent comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1,

and wherein the compound of structural formula (I) has a GWP (100 year ITH) of less than 2000.

8. A device comprising:

a dielectric fluid comprising a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1;

wherein the device is an electrical device.

9. The device of embodiment 8, wherein said electrical device comprises gas-insulated circuit breakers, current-interruption equipment, a gas-insulated transmission line, gas-insulated transformers, or a gas-insulated substation.
10. The device according to any one of embodiments 8-9, wherein the dielectric fluid further comprises a second dielectric fluid.
11. The device according to embodiment 10, wherein the second dielectric fluid comprises an inert gas.
12. The device according to any one of embodiments 10-11, wherein the second dielectric fluid comprises air, nitrogen, nitrous oxide, oxygen, helium, argon, carbon dioxide, heptafluoroisobutyronitrile, 2,3,3,3-tetrafluoro-2-(trifluoromethoxy)propanenitrile, 1,1,1,3,4,4,4-heptafluoro-3-(trifluoromethyl)butan-2-one, SF₆, or combinations thereof.
13. The device according to any one of embodiments 8-12, wherein R¹, R², and R³ are perfluorinated.
14. The device according to any one of embodiments 8-13, wherein, n=0 and R¹ and R² are each independently a fluoroalkyl group having from 1 to 2 carbon atoms
15. The device according to any one of embodiments 8-14, wherein the compound of structural formula (I) has a GWP (100 year ITH) of less than 2000.
16. An apparatus for converting thermal energy into mechanical energy in a Rankine cycle comprising:

a working fluid;

a heat source to vaporize the working fluid and form a vaporized working fluid;

a turbine through which the vaporized working fluid is passed thereby converting thermal energy into mechanical energy;

a condenser to cool the vaporized working fluid after it is passed through the turbine; and

a pump to recirculate the working fluid,

wherein the working fluid comprises a compound having structural formula (I)

R¹SO₂R²(SO₂R³)ₙ (I)

where R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

17. The apparatus according to embodiment 16, wherein the compound is present in the working fluid at an amount of at least 25% by weight based on the total weight of the working fluid.
18. The apparatus according to any one of embodiments 16-17, wherein R¹, R², and R³ are perfluorinated.
19. The apparatus according to any one of embodiments 16-18, wherein the compound of structural formula (I) has a GWP (100 year ITH) of less than 2000.
20. A process for converting thermal energy into mechanical energy in a Rankine cycle comprising:

vaporizing a working fluid with a heat source to form a vaporized working fluid;

expanding the vaporized working fluid through a turbine;

cooling the vaporized working fluid using a cooling source to form a condensed working fluid; and

pumping the condensed working fluid;

wherein the working fluid comprises a a compound having structural formula (I)

R¹SO₂R²(SO₂R³)ₙ (I)

where R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1,

and wherein the compound of structural formula (I) has a GWP (100 year ITH) of less than 2000.

21. A process for recovering waste heat comprising:

passing a liquid working fluid through a heat exchanger in communication with a process that produces waste heat to produce a vaporized working fluid;

removing the vaporized working fluid from the heat exchanger;

passing the vaporized working fluid through an expander, wherein the waste heat is converted into mechanical energy; and

cooling the vaporized working fluid after it has been passed through the expander;

wherein the working fluid comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1,

and wherein the compound of structural formula (I) has a GWP (100 year ITH) of less than 2000.

22. An immersion cooling system comprising:

a housing having an interior space;

a heat-generating component disposed within the interior space; and

a working fluid liquid disposed within the interior space such that the heat-generating component is in contact with the working fluid liquid;

wherein the working fluid comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

23. The system according to embodiment 22, wherein the compound is present in the working fluid at an amount of at least 25% by weight based on the total weight of the working fluid.
24. The system according to any one of embodiments 22-23, wherein R¹, R², and R³ are perfluorinated.
25. The system according to any one of embodiments 22-24, wherein the heat-generating component comprises an electronic device.
26. The system according to any one of embodiments 22-25, wherein the electronic device comprises a computer server.
27. The system of embodiment 26, wherein the computer server operates at frequency of greater than 3 GHz.
28. The system according to any one of embodiments 22-27, wherein the immersion cooling system further comprises a heat exchanger disposed within the system such that upon vaporization of the working fluid liquid, the working fluid vapor contacts the heat exchanger;
29. The system according to any one of embodiments 22-28, wherein the immersion cooling system comprises a two-phase immersion cooling system.
30. The system according to any one of embodiments 22-29, wherein the immersion cooling system comprises a single-phase immersion cooling system.
31. The system according to any one of embodiments 22-30, wherein the immersion cooling system further comprises a pump that is configured to move the working fluid to and from a heat exchanger.
32. The system according to any one of embodiments 22-31, wherein the compound of structural formula (I) has a GWP (100 year ITH) of less than 2000.
33. A method for cooling a heat generating component, the method comprising:

at least partially immersing a heat generating component in a working fluid; and

transferring heat from the heat generating component using the working fluid;

wherein the working fluid comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1;

and wherein the compound of structural formula (I) has a GWP (100 year ITH) of less than 2000.

34. A thermal management system for a lithium-ion battery pack comprising:

a lithium-ion battery pack; and

a working fluid in thermal communication with the lithium-ion battery pack;

wherein the working fluid comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

35. The system according to embodiment 34, wherein the compound is present in the working fluid at an amount of at least 25% by weight based on the total weight of the working fluid.
36. The system according to any one of embodiments 34-35, wherein R¹, R², and R³ are perfluorinated.
37. The system according to any one of embodiments 34-36, wherein the compound of structural formula (I) has a GWP (100 year ITH) of less than 2000.
38. A thermal management system for an electronic device, the system comprising:

an electronic device selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, or a laser; and

a working fluid in thermal communication with the electronic device;

wherein the working fluid comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

39. The thermal management system according to embodiment 38, wherein the device is selected from a microprocessor, a semiconductor wafer used to manufacture a semiconductor device, a power control semiconductor, a circuit board, a multi-chip module, or a packaged or unpackaged semiconductor device.
40. The thermal management system according to any one of embodiments 38-39, wherein the electronic device is at least partially immersed in the working fluid.
41. The thermal management system according to any one of embodiments 38-40, wherein the compound of structural formula (I) has a GWP (100 year ITH) of less than 2000.
42. A system for making reactive metal or reactive metal alloy parts comprising:

a molten reactive metal is selected from magnesium, aluminum, lithium, calcium, strontium, and their alloys; and

a cover gas disposed on or over a surface of the molten reactive metal or reactive metal alloy;

wherein the cover gas comprises a compound having structural formula (I)

R¹SO₂R²(SO₂R³)ₙ (I)

where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1,

and wherein the compound of structural formula (I) has a GWP (100 year ITH) of less than 2000.

43. A system for making reactive metal or reactive metal alloy parts according to embodiment 42, wherein the molten reactive metal comprises magnesium or a magnesium alloy.
44. A system according to any one of embodiments 42-43, wherein R², and R³ are perfluorinated.

### EXAMPLES

Objects and advantages of this disclosure are further illustrated by the following comparative and illustrative examples. Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, Mo., US, or may be synthesized by conventional methods. The following abbreviations are used herein: mL=milliliters, L=liters, min=minutes, hr=hours, g=grams, μm=micrometers (10⁻⁶ m), ° C.=degrees Celsius, cSt=centi Stokes, KHz=kilohertz, kV=kilovolts, J=Joules, ppm=parts per million, kPa=kiloPascals, K=degrees Kelvin.

### Example 1: Perfluorodimethylsulfone, CF₃SO₂CF₃

A dry 600 ml pressure reactor was charged with 100 grams anhydrous acetonitrile, 56.1 grams (0.39 moles) trimethyl(trifluoromethyl) silane and 2.5 grams (0.04 moles) anhydrous potassium fluoride. The reactor was cooled in dry ice and evacuated. 50 grams (0.33 moles) of perfluoromethanesulfonyl fluoride (available from the process described in EP0707094B1, Example 1) was charged to the reactor and contents allowed to come to room temperature with stirring. The reactor was held at 25° C. for an additional 2 hours and the vapor space was condensed into a −70° C. evacuated, stainless steel cylinder. 68 grams were recovered with a perfluorodimethyl sulfone purity of 19.4% by GC-FID. The perfluorodimethyl sulfone can be further purified by water washing and fractional distillation. The boiling point was approximately 15° C. The identity and purity of the product was confirmed by GC-MS and ¹⁹F NMR spectroscopy.

### Example 2: 1,1,1,2,2,3,3,4,4-nonafluoro-4-((trifluoromethyl)sulfonyl)butane, CF₃SO₂C₄F₉

To a three neck, 500 mL round-bottom flask equipped with a magnetic stir bar, temperature probe, and water-cooled reflux condenser was charged CsF (14.1 g, 92.8 mmol). The reaction vessel was evacuated and back-filled with nitrogen gas three times followed by the addition of anhydrous diglyme (125 mL) and 1,1,2,2,3,3,4,4,4-nonafluorobutane-1-sulfonyl fluoride (170 g, 563 mmol). The resultant mixture was stirred at room temperature followed by the dropwise addition of trimethyl(trifluoromethyl)silane (88.0 g, 619 mmol) over the course of 3 hours. The rate of addition was such that the internal reaction mixture did not exceed 36° C. After complete addition, the resultant reaction mixture was allowed to stir for 16 hours with no heating followed by the addition water (300 mL). The fluorous phase was collected and the resultant crude product mixture was analyzed by GC-FID, which indicated complete conversion of the trimethyl(trifluoromethyl)silane. Concentric tube distillation of the fluorous phase afforded the desired 1,1,1,2,2,3,3,4,4-nonafluoro-4-((trifluoromethyl)sulfonyl)butane (95° C., 740 mm/Hg, 78 g, 39% yield) as a colorless liquid. The identity and purity of the product was confirmed by GC-MS analysis.

### Example 3: Perfluorodiethylsulfone, C₂F₅SO₂C₂F₅

A dry 4.0 L pressure reactor was charged with 50.0 g KF, 1,500.0 g DMF, 100.0 g 18-crown-6, and 1.0 g alpha-pinene, and immediately sealed up to minimize exposure to atmospheric moisture. After removing residual oxygen at −20° C. under vacuum, the reactor was charged with 400 g of SO₂F₂ (available from Douglas Products, Liberty, Mo., US). The reactor was then warmed to 70° C. and tetrafluorethylene (TFE, available from ABCR GmbH, Karlsruhe, Germany) was charged at 200 g/hr until a total of 800 g total TFE was charged to the reactor. Once all the TFE was charged, the reactor temperature was increased to 90° C. and held at this temperature with agitation until the drop-in reactor pressure leveled off, indicating that reaction was near completion. Then the temperature was decreased to −20° C. and the reactor was briefly evacuated to remove residual unreacted TFE and SO₂F₂. Vacuum was relieved with nitrogen and the reactor was warmed to room temperature and the contents were drained and collected. The crude reaction mixture consisted of two non-miscible liquid phases along with some suspended KF. The reaction mixture was transferred to a separatory funnel, combined with 1.5 kg of water and shaken. The two-phase mixture was allowed to phase separate and the lower fluorochemical phase was collected and washed with three 1.0 Kg portions of water. After the final water wash, the lower fluorochemical phase was collected (911.0 g), and passed through a short column of silica gel 60 (70-230 mesh) to remove color and residual moisture. The eluent was then purified by fractional distillation using a 20-tray Oldershaw column at atmospheric pressure yielding approximately 680 g of pure perfluorodiethylsulfone (99.85% pure by GC-FID). The identity and purity of the product was confirmed by GC-MS and ¹⁹F NMR spectroscopy.

### Example 4: 1,1,1,2,2,3,3,4,4-nonafluoro-4-((perfluoroethyl)sulfonyl)butane, C₂F₅SO₂C₄F₉

To a 3-neck round bottom flask equipped with a stir bar, water-cooled reflux condenser, and temperature probe was charged CsF (2.51 g, 16.6 mmol). The reaction vessel was evacuated and back-filled with nitrogen gas three times followed by the addition of anhydrous tetraglyme (75 mL) and 1,1,2,2,3,3,4,4,4-nonafluorobutane-1-sulfonyl fluoride (50.2 g, 166 mmol). The resultant mixture was stirred at room temperature followed by the dropwise addition of trimethyl(perfluoroethyl)silane (39.1 g, 203 mmol) over the course of 2 hours. The rate of addition was such that the internal reaction mixture temperature did not exceed 41° C. After complete addition, the resultant reaction mixture was allowed to stir for 16 hours with no heating followed by the addition water (100 mL). The fluorous phase was collected and analyzed by GC-FID, which indicated complete conversion of the trimethyl(perfluoroethyl)silane starting material. Concentric tube distillation of the fluorous phase afforded 47.4 g (71% yield) of the desired 1,1,1,2,2,3,3,4,4-nonafluoro-4-((perfluoroethyl)sulfonyl)butane as a colorless liquid (B.P.=118° C., 740 mm/Hg, Purity=97.4% by GC-FID uncorrected for response factors). The identity and purity of the product was confirmed by GC-MS analysis.

### Example 5: Perfluorodimethylsulfone, CF₃SO₂CF₃

A Simons electrochemical fluorination (ECF) cell of essentially the type described in U.S. Pat. No. 2,713,593 was used to electrochemically fluorinate dimethyl sulfone, CH₃SO₂CH₃. The crude fluorinated product was treated with sodium fluoride to remove dissolved hydrogen fluoride, then fractionally distilled in a 44-tray vacuum jacketed Oldershaw column. The boiling point of the product cut was approximately 15° C. The combined product cuts totaled 413.9 grams of distilled product. GC-MS/TCD analysis of the product was reported as 98.0 area % perfluorodimethylsulfone, CF₃SO₂CF₃.

### Example 6: 1,1,1,2,2-pentafluoro-2-((trifluoromethyl)sulfonyl)ethane, CF₃SO₂CF₂CF₃)

To a 2 L stainless steel reaction vessel were charged cesium fluoride (56.4 g, 371 mmol) and tetraglyme (500 g). The vessel was then evacuated and charged with perfluoroethanesulfonyl fluoride (500 g, 2.47 mol). To the resultant stirring mixture was slowly added trimethyl(trifluoromethyl)silane (387 g, 2.72 mol) over the course of one hour via a stainless steel cylinder pressurized with argon. After complete addition, the resultant reaction mixture was allowed to stir overnight at room temperature. The internal temperature was then raised to approximately 70° C. and the headspace was transferred to an evacuated stainless steel cylinder submerged in a dry ice/acetone bath. GC-FID analysis of the crude mixture indicated complete conversion of the perfluoroethanesulfonyl fluoride. The contents of the stainless steel cylinder were transferred to a round bottom flask and were then purified via concentric tube distillation to afford the desired 1,1,1,2,2-pentafluoro-2-((trifluoromethyl)sulfonyl)ethane (120 g at 92% purity, 18% isolated yield) as a colorless liquid. The identity and purity of the product were confirmed by GC-MS analysis.

### Physical Properties

Properties of Examples 2, 3, 4, and 5 were measured and compared with other fluorinated fluids commonly used in immersion cooling applications: Comparative Example CE1 (NOVEC 7100, available from 3M, St. Paul, Minn., US), CE2 (NOVEC 7300, available from 3M, St. Paul, Minn., US), CE3 (OPTEON SF10, an unsaturated hydrofluoroether, available from Chemours, Wilmington, Del., US), CE4 (FLUORINERT FC-3283, a perfluorinated amine (PFA) available from 3M, St. Paul, Minn., US) and CE5 (GALDEN HT-110, a perfluorinated polyether (PFPE) available from Solvay, Brussels, Belgium).

Kinematic viscosities were measured using a Schott AVS 350 Viscosity Timer. For temperatures below 0° C., a Lawler temperature control bath was used. The viscometers used for all temperatures were Ubbelohde capillary viscometers type numbers 545-03, 545-10, 545-13 and 545-20. Viscometers were corrected using the Hagenbach correction.

Boiling points were measured according to the procedures in ASTM D1120-94 “Standard Test Method for Boiling Point of Engine Coolants.”

Pour points were determined by placing approximately 2 mL of the sample in a 4 mL glass vial into a manually temperature controlled bath. Temperature was read with Analytical Instrument No. 325. Pour point is defined as the lowest temperature at which, after being tilted horizontally for 5 seconds, the sample is visually observed to flow.

The dielectric constants and electrical dissipation factors (tan delta) were measured using an Alpha-A High Temperature Broadband Dielectric Spectrometer (Novocontrol Technologies, Montabaur, Germany) in accordance with ASTM D150-11, “Standard Test Methods for AC Loss Characteristics and Permittivity (Dielectric Constant) of Solid Electrical Insulation.” The parallel plate electrode configuration was selected for this measurement. The sample cell of parallel plates, an Agilent 16452A liquid test fixture consisting of 38 mm diameter parallel plates (Keysight Technologies, Santa Rosa, Calif., US) was interfaced to the Alpha-A mainframe while utilizing the ZG2 Dielectric/Impedance General Purpose Test Interface (available from Novocontrol Technologies, Montabaur, Germany). Each sample was prepared between parallel plate electrodes with a spacing, d, (typically, d=1 mm) and the complex permittivity (dielectric constant and loss) were evaluated from the phase sensitive measurement of the electrodes voltage difference (Vs) and current (Is). Frequency domain measurements were carried out at discrete frequencies from 0.00001 Hz to 1 MHz. Impedances from 10 milliOhms up to 1×10¹⁴ ohms were measured up to a maximum of 4.2 volts AC. For this experiment, however, a fixed AC voltage of 1.0 volts was used. The DC conductivity (the inverse of volume resistivity) can also be extracted from an optimized broadband dielectric relaxation fit function that contains at least one term of the low frequency Havrrilak Negami dielectric relaxation function and one separate frequency dependent conductivity term.

The liquid dielectric breakdown strength measurements were performed in accordance with ASTM D877-87(1995) Standard Test Method for Dielectric Breakdown Voltage of Insulating Liquids. Disk electrodes 25 mm in diameter were utilized with a Phenix Technologies Model LD 60 that is specifically designed for testing in the 7-60 kV, 60 Hz (higher voltage) breakdown range. For this experiment, a frequency of 60 Hz and a ramp rate of 500 volts per second were utilized, as is typical.

Heats of vaporization were calculated from the vapor pressure curves of the respective fluids using the Clausius-Clapeyron equation:

dHᵥₐₚ(Joules per mole)=d(ln(Pᵥₐₚ))/d(1/T)×R

where R is the universal gas constant (8.314 Joules per mol per ° C.). Vapor Pressure as a function of temperature was measured using the stirred-flask ebuilliometer method described in ASTM E-1719-97 “Vapor Pressure Measurement by Ebuilliometry” and the data collected was used to construct vapor pressure curves.

Environmental lifetimes and Global Warming Potential (GWP) values were determined using methods described in Intergovernmental Panel on Climate Change (IPCC) Fifth Assessment Report (AR5) that consists of essentially three parts:
(1) Calculation of the radiative efficiency of the compound based upon a measured infrared cross-section for the compound.
(2) Calculation, measurement, or estimation of the atmospheric lifetime of the compound.
(3) Combination of the radiative efficiency and atmospheric lifetime of the compound relative to that of CO₂ over a time horizon of 100 years.
   The three steps used to calculate a GWP were as follows. A gas standard of the material to be assessed, having a known and documented concentration was prepared at the 3M Environmental Lab and used to obtain the FTIR spectra of this compound. Quantitative gas phase, single component FTIR library reference spectra were generated at two different concentration levels by diluting the sample standard with nitrogen using mass flow controllers. The flow rates were measured using certified BIOS DRYCAL flow meters (Mesa Labs, Butler, N.J., US) at the FTIR cell exhaust. The dilution procedure was also verified using a certified ethylene calibration gas cylinder. Using methods described in AR5, the FTIR data was used to calculate the radiative efficiency, which in turn was combined with the atmospheric lifetime to determine the global warming potential (GWP) value.

A Global Warming Potential (GWP) value was determined for Examples 3, 4, and 5) using the three part method AR5 described previously, as detailed below for Example 3. The radiative efficiency of Example 3 (perfluorodiethylsulfone) was calculated to be 0.282 Wm⁻² ppbV⁻¹. This radiative efficiency takes into account stratospheric temperature adjustment and lifetime correction. The atmospheric lifetime of perfluorodiethylsulfone was determined from relative rate studies utilizing chloromethane (CH₃Cl) as a reference compound. The pseudofirst order reaction rates of the reference compound and perfluorodiethylsulfone with hydroxyl radicals (.OH) was determined in a laboratory chamber system. The atmospheric lifetime of the reference compound is documented in the literature, and based on this value and the pseudo first order rates measured in the chamber experiments, the atmospheric lifetime for Example 3 (perfluorodiethylsulfone) was determined to be 10 years. The concentrations of gases in the test chamber were quantified by FTIR. The measured atmospheric lifetime value of Example 3 was used for GWP calculation. The resulting 100-year GWP value for Example 3 (perfluorodiethylsulfone) was determined to be 580. The GWP values for Example 4 and 5 were determined via an analogous process.

The physical properties and environmental lifetime results for Examples 2, 3, 4, and 5 and CE1-CE5 are summarized in Table 1 and illustrate that the perfluorinated sulfones in general, and perfluorodiethylsulfone in particular, provide superior dielectric properties (lower dielectric constant, higher or comparable dielectric strength, higher volume resistivity) than the comparative hydrofluoroethers CE1-CE3. Table 1 also illustrates that Examples 3, 4, and 5 surprisingly have a much lower environmental lifetime and global warming potential than CE4 (a PFA) and CE5 (a PFPE). The results further show that Example 3 provides a significantly higher heat of vaporization than any of the other Comparative Examples, a property that is critical to two-phase immersion cooling performance for electronics or batteries. Finally, the results show that Examples 3 and 4 provide comparable (or superior) low temperature properties, as measured by pour point and temperature dependent viscosity, compared to the comparative fluids—another important factor in immersion cooling performance.

TABLE 1
Physical Properties

| | Ex. 2 | Ex. 3 | Ex. 4 | Ex. 6 | CE1 | CE2 | CE3 | CE4 | CE5 |
|---|---|---|---|---|---|---|---|---|---|
| Boiling Point (° C.) | 95 | 64 | 112.4 | 39 | 61 | 98 | 110 | 128 | 110 |
| Pour Point (° C.) | | −86 | −84 | | −135 | −38 | <−90 | −50 | −100 |
| Kinematic | | 0.34 | 0.72 | | 0.61 | 0.7 | 0.7 | 0.8 | 0.8 |
| Viscosity @ 25° C. (cSt) | | | | | | | | | |
| Kinematic | | 1.17 | 3.4 | | — | — | 3.4 | 5.3 | 3.7 |
| Viscosity @ −40° C. (cSt) | | | | | | | | | |
| Dielectric | 3.3 | 3.23 | 3.0 | | 7.4 | 6.1 | 5.5 | 1.9 | 1.92 |
| Constant (1 KHz) | | | | | | | | | |
| Liquid Dielectric | | 32 | | | 25 | 34 | 29 | 43 | 40 |
| Breakdown | | | | | | | | | |
| Strength (kV) | | | | | | | | | |
| Volume | | 1.9 × 10¹² | | | 10⁸ | 10¹¹ | 10¹⁰ | 10¹⁵ | 5 × 10¹⁵ |
| Resistivity | | | | | | | | | |
| (ohm-cm) | | | | | | | | | |
| Heat of | | 123 | | | 112 | 102 | 115 | 78 | 71 |
| Vaporization | | | | | | | | | |
| (J/g at 25° C.) | | | | | | | | | |
| Atmospheric | | 10 | 10 | 8.2 | 4.1 | 3.8 | <0.03 | 2,000 | — |
| Lifetime (yr) | | | | | | | | | |
| GWP (100 Year) | | 580 | 550 | 647 | 297 | 310 | 2.5 | 8,690 | >8,000 |

### Heat Transfer Coefficient

The heat transfer apparatus used for the measurement of change in heat transfer coefficient (HTC) as a function of heat flux comprised a phenolic platform containing a 25-mm diameter copper heater atop 4 thin radial ribs. A thermocouple probe integrated into the platform above the heater was placed so that a greased boiling enhancement coating (BEC) disk could be placed onto the probe and atop the heater. The BEC, obtained from Celsia, Santa Clara, Calif., US with an identification number of 01MMM02-A1, had a thickness of 300 was comprised of 50 μm particles, and was coated in a 5 cm² area on a 3-mm thick, 100 series copper disk. The thermocouple probe was bent in such a way that when the disk was locked down into the proper x-y position, the probe was gently pressed upward and into the termination of the thermocouple groove to measure the sink temperature (Tₛ). The platform moved on z-axis sliders with a lever and spring that engaged the BEC disk to a gasketed glass tube into which another thermocouple protruded to measure T_{f}, the fluid saturation temperature.

Approximately 10 mL of fluid was added through a fill port at the top of the apparatus. Vapor was condensed in an air-cooled condenser and allowed to fall back into the pool. The condenser was open at the top so that P=Pₐₜₘ and T_{f}=T_{b}=Tₛ(Pₐₜₘ). Measurements began with a 3-min warm-up at 100 W (20 W/cm²) intended to minimize conduction losses from the bottom of the copper heater during subsequent measurements. The power was then lowered to 50 W (10 W/cm²) and allowed to equilibrate for 2 min at which time data were recorded before advancing 10 W to the next data point. This continued until Tₛ exceeded a preset limit, usually about T_{b}+20° C. The data acquisition system queried the DC power supply for the heater voltage, V, and current, I. The heat flux, Q″, and heat transfer coefficient, H, are defined as Q″=Q/A=VI/A and H=Q″/(Tₛ−T_{f}), where A is area.

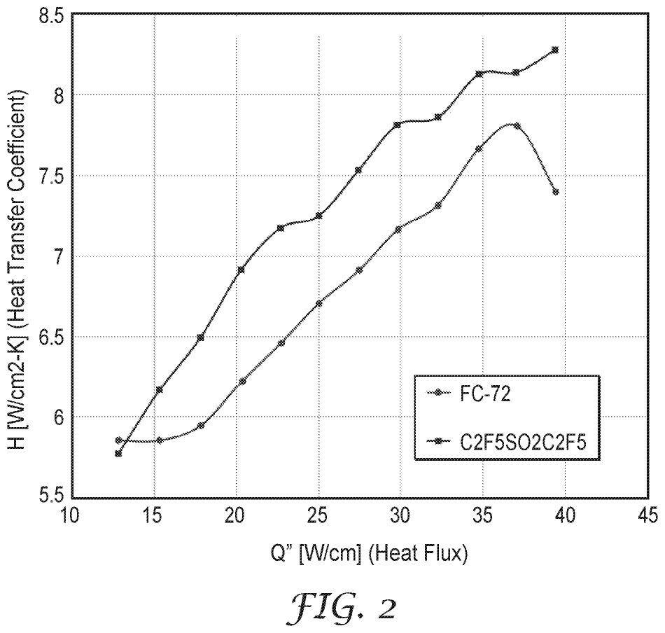

The heat transfer coefficient of perfluorodiethylsulfone (Example 3) was measured as a function of heat flux and compared to Comparative Example CE6 (FLUORINERT FC-72, a perfluorocarbon (PFC) available from 3M, St. Paul, Minn., US.) The results are plotted in FIG. 2. For use in two-phase immersion cooling, higher heat transfer coefficients are preferred. Thus, the data in FIG. 2 shows that Example 3 has improved heat transfer properties for two phase immersion cooling applications compared to a commonly used heat transfer fluid, CE6, while also providing the environmental benefits of a much lower global warming potential than CE6.

### Gas Phase Dielectric Breakdown Voltage

The gaseous dielectric breakdown strength of perfluorodiethylsulfone (Example 3) and perfluorodimethylsulfone (Example 5) and comparative examples CE7 (SF₆, available from Solvay, Brussels, Belgium) and CE8 (perfluorocyclopropane, cyclo-C₃F₆, available from SynQuest Laboratories, Alachua, Fla., US) were measured experimentally using a Hipotronics OC60D dielectric strength tester (available from Hipotronics, Brewster, N.Y.). A gas-tight cell was constructed from PTFE using parallel disk electrodes similar to those described in ASTM D877-13, “Standard Test Method for Dielectric Breakdown Voltage of Insulating Liquids Using Disk Electrodes.” The test cell was first evacuated and the dielectric breakdown voltage was measured as increasing pressures of gaseous test compound were added to the cell. The dielectric breakdown voltage was measured 10 times after each addition of gas.

The average values of the 10 measurements at each pressure are summarized in Tables 2A and 2B. Surprisingly, the results illustrate that perfluorodiethylsulfone (Example 3) and perfluorodimethylsulfone (Example 5) provide significantly higher dielectric breakdown strength than SF₆ (CE7), a commercial dielectric gas widely used in gas insulated high voltage switch gear and transmission power lines at equivalent absolute pressures. Perfluorodiethylsulfone (Example 3) also demonstrated significantly higher dielectric breakdown strength than perfluorocyclopropane (CE8, a PFC that has been considered for use in similar applications) at equivalent absolute pressures. Furthermore, Examples 3 and 5 provide this improved gas phase dielectric breakdown performance while also providing more than a factor of 10 lower GWP than either of the comparative materials, as shown previously in Table 1.

TABLE 2A
Gas Phase Dielectric Breakdown Voltage of
Perfluorodiethylsulfone, SF₆ and Cyclo-C₃F₆

| Example 3 | | CE7 | | CE8 | |
|---|---|---|---|---|---|
| Absolute | | Absolute | | Absolute | |
| Pressure | average | Pressure | average | Pressure | average |
| (kPa) | kV | (kPa) | kV | (kPa) | kV |
| 6.9 | 4.5 | 13.9 | 4.6 | 13.8 | 4.6 |
| 13.4 | 6.5 | 27.6 | 5.4 | 27.9 | 6.4 |
| 28.1 | 9.1 | 41.4 | 7.8 | 41.4 | 8.0 |

TABLE 2B
Gas Phase Dielectric Breakdown Voltage
of Perfluorodimethylsulfone and SF₆

| Example 5 | | CE7 | |
|---|---|---|---|
| Absolute Pressure | | Absolute Pressure | |
| (kPa) | average kV | (kPa) | average kV |
| 50 | 11.2 | 55 | 9.5 |
| 75 | 14.7 | 69 | 10.9 |
| 100 | 18.1 | 83 | 12.5 |
| 125 | 21.1 | 97 | 13.5 |
| 150 | 24.1 | 110 | 15.3 |
| 175 | 26.5 | 124 | 16.7 |
| 190 | 28.6 | 139 | 18.0 |
| 208 | 30.3 | 152 | 19.2 |
| 50 | 11.2 | 55 | 9.5 |
| 75 | 14.7 | 69 | 10.9 |
| 100 | 18.1 | 83 | 12.5 |

### Thermal and Hydrolytic Stability

### Thermophysical Properties

Table 3 illustrates that Examples 3, 4, and CE1 have similar thermophysical properties.

TABLE 3
Thermophysical Properties

| | Normal | | | Vapor | Specific |
|---|---|---|---|---|---|
| | Boiling | Pour | Viscosity | Pressure | Heat |
| | Point | Point | @ 25° C. | @ 25° C. | Capacity |
| | (° C.) | (° C.) | (×10⁻⁷ m²/s) | (kPa) | (J/kg-K) |
| Example 3 | 64 | −86 | 3.4 | 23 | 1181 |
| Example 4 | 112.4 | −84 | | | 1060 |
| CE1 | 61 | −135 | 3.8 | 27 | 1183 |

### Hydrolytic Stability

Duplicate samples of Example 3 and CE1 were tested for hydrolytic stability at 150° C. by placing 10 grams of test material along with 10 grams deionized water in a clean, 40 mL Monel pressure vessel, which was sealed and placed in a convection oven set at 150° C. for 24 hours. After aging, the fluoride concentrations were determined by mixing 1 mL of the water phase from each sample with 1 mL of TISAB II (Total Ionic Strength Buffer) buffer solution. Fluoride ion concentrations were then measured using an ORION EA 940 meter with an ORION 9609BNWB Fluoride-Ion Specific Electrode (ISE) (Thermo Fisher Scientific, Minneapolis, Minn., US). ORION IONPLUS Fluoride standards (1, 2, 10 and 100 ppm Fluoride) were used for the calibration of the meter.

The hydrolytic stability values of Example 3 and CE1 are reported as average parts per million by weight (ppmw) of free fluoride in water in Table 4. Higher levels of free fluoride ion concentration correspond to reduced stability. Results show that the hydrolytic stability of perfluorodiethylsulfone (Example 3) is significantly better than Comparative Example CE1.

TABLE 4
Hydrolytic Stability

| | | Average F concentration |
|---|---|---|
| | Average F concentration | after 24 hours at |
| | at room temperature | 150° C. with DI H₂O |
| Sample | (ppmw) | (ppmw) |
| CE1 | <0.05 | 395.0 |
| Example 3 | <0.05 | 11.55 |

### Thermal Stability

The thermal stability of Example 3 and CE1 was determined by placing duplicate 10-gram samples in clean, 40 mL Monel pressure vessels and sealing tightly. The pressure vessels were then placed in a convection oven set at 100° C. for 24 hours. After aging, each sample was mixed with a known weight of ultrapure (18.2 MΩ) water, agitated in a mechanical shaker at high speed for 15 minutes and finally centrifuged to separate the two phases. Fluoride ion concentrations were subsequently measured in the water phase as previously described. This was then followed by another experiment at 150° C. using the same method. The fluoride ion concentrations measured for Example 3 and CE1 were both less than 0.5 ppmw at 100 and 150° C., as shown in Table 5, indicating that these materials both provide excellent thermal stability in the absence of water.

TABLE 5
Thermal Stability

| | Average F | Average F | Average F |
|---|---|---|---|
| | concentration at | concentration after | concentration after |
| | room temperature | 24 hours at 100° C. | 24 hours at 150° C. |
| Sample | (ppmw) | (ppmw) | (ppmw) |
| CE1 | <0.05 | <0.05 | 0.05 |
| Example 3 | <0.05 | <0.05 | 0.36 |

### Use as Working Fluid in Organic Rankine Cycle

The critical temperature and pressure of Example 3 (presented in Table 6) were determined from its molecular structure using the method of Wilson-Jasperson given in Poling, Prausnitz, O'Connell, The Properties of Gases and Liquids, 5^{th} ed., McGraw-Hill, 2000.

The critical density was estimated using a generalized liquid density correlation from Valderrama, J. O; Abu-Shark, B., Generalized Correlations for the Calculation of Density of Saturated Liquids and Petroleum Fractions. Fluid Phase Equilib. 1989, 51, 87-100. Inputs for the correlation were the measured normal boiling point, liquid density at 25° C. and estimated critical temperature from above.

Ideal gas heat capacity was calculated from measured liquid heat capacity, using the corresponding states equation for liquid specific heat given in Poling, Prausnitz, O'Connell, The Properties of Gases and Liquids, 5^{th} ed., McGraw-Hill, 2000.

Thermodynamic properties for Example 3 were derived using the Peng-Robinsion equation of state (Peng, D. Y., and Robinson, D. B., Ind. & Eng. Chem. Fund. 15: 59-64, 1976.) Inputs required for the equation of state were critical temperature, critical density, critical pressure, acentric factor, molecular weight and ideal gas heat capacity.

For CE1, thermophysical property data were fitted to a Helmholtz equation of state, with the functional form described in Lemmon E. W., Mclinden M. O., and Wagner W., J. Chem. & Eng. Data, 54: 3141-3180, 2009.

TABLE 6
Thermophysical Properties

| | | Specific Heat | Critical | Critical | Critical |
|---|---|---|---|---|---|
| | | Capacity | Temperature | Pressure | Density |
| | Material | (J/kg-K) | (° C.) | (kPa) | (kg/m³) |
| CE1 | C₄F₉OCH₃ | 1183 | 195 | 2230 | 555 |
| Ex. 3 | C₂F₅SO₂C₂F₅ | 1181 | 183 | 2040 | 434 |

A Rankine cycle based on the configuration of FIG. 3, and operating between 50° C. and 140° C., was used to assess the performance of both Example 3 and CE1. The Rankine cycle was modeled using the calculated thermodynamic properties from the equations of state and the general procedure described in Cengel Y. A. and Boles M. A., Thermodynamics: An Engineering Approach, 5^{th} Edition; McGraw Hill, 2006. The heat input for the cycle was 1000 kW, with working fluid pump and expander efficiencies taken to be 60% and 80% respectively. Results are shown in Table 7. The thermal efficiency of perfluorodiethylsulfone (Example 3) was calculated to be comparable to CE1.

TABLE 7
Calculated Rankine Cycle Performance

| | Example 3 | CE1 |
|---|---|---|
| Condenser Temperature [° C.] | 50.0 | 50.0 |
| Condenser Pressure [kPa] | 62 | 71 |
| Boiler Temperature [° C.] | 140 | 140 |
| Boiler Pressure [kPa] | 860.1 | 829.2 |
| Fluid Flow [kg/s] | 5.3 | 5.0 |
| Pump Work [kJ/kg] | 0.81 | 0.87 |
| Q, Boiler [kJ/kg] | 188.3 | 200.3 |
| Expander Work [kJ/kg] | 20.6 | 23.0 |
| Net Work [kJ/kg] | 19.8 | 22.1 |
| Net Work [kW] | 105.1 | 110.5 |
| Thermal Efficiency | 0.105 | 0.110 |

### Inhalation Toxicity in Rats

The inhalation toxicity potential of Example 3 was evaluated in male Sprague Dawley rats after a single 4-hour whole body exposure at atmospheric concentrations of 10,000 ppm (v/v). The test material (purity 98.84%) was administered as received at an appropriate volume to a 40-L test chamber containing 3 rats. The test material vaporized upon addition to the chamber. The air within the chamber was regenerated at appropriate intervals to maintain an 18% oxygen concentration. Three control animals were placed in another chamber filled with ambient air. The day of exposure was designated Day 0. Clinical observations were recorded during the exposure period and for 14 days after exposure. Body weights were recorded prior to exposure (Day 0), on Day 1, Day 2, and 14 days after exposure for both the test material-treated and control animals. There was no mortality or abnormal clinical observations reported during the 4-hour exposure period and throughout the 14-day study. All animals gained weight and were normal throughout the study period and at gross necropsy. Similar results were obtained in a 3 day inhalation repeat dose study conducted at the same dose level. In conclusion, based on the results of this study, the approximate inhalation 4-hour LC₅₀ of perfluorodiethylsulfone (Example 3) is greater than 10,000 ppm.

### Stability as a Foam Additive in Polyol-Amine Catalyst Mixture

The stability of Example 3 (perfluorodiethylsulfone) was measured in a standard polyol/amine catalyst/foam blowing agent mixture commonly used in making polyurethane foams. The stability was compared to CE9 (PF-5060) and CE10 (FA-188), both of which are available from 3M Company, St. Paul, Minn., US. Stability was determined by measuring the increase in fluoride ion levels over time after mixing all the components at room temperature. An increase in fluoride ion levels is a measure of the extent to which the fluorinated foam additive is reacting with the polyol/amine catalyst mixture to release fluoride ion. Fluoride ion measurements were made using a ThermoScientific ORION DUAL STAR pH/ISE channel meter and VWR 14002-788 F Fluoride specific electrode. The electrode was calibrated using fluoride standards of 1, 2, 10, and 100 ppm fluoride ion concentration in aqueous TISAB II (Total Ionic Strength Adjustment Buffer) buffer solution.

The Polyol/Amine Catalyst/Blowing Agent/Foam Additive sample mixtures were prepared by mixing ELASTAPOR P 17655R Resin (a polyol/amine catalyst blend obtained from BASF, Ludwigshafen, Germany), cyclopentane (a common foam blowing agent), and Example 3, CE9, or CE10 as a foam additive. Using a SARTORIUS A200S balance, the cyclopentane/foam additive mixtures were made first by mixing 25.5 grams of cyclopentane with 2.3 grams of foam additive. Then 43.1 grams of the ELASTAPOR polyol containing the amine catalyst was transferred to a wide mouth 4 oz glass jar and 7 g of the cyclopentane/foam additive mixture was added and shaken.

After the sample mixtures were thoroughly shaken and mixed, an aliquot was removed and an initial fluoride concentration was determined at time 0 hr. Analytical samples were prepared by diluting 1 g of the sample mixture with 1 g of isopropyl alcohol and 0.5 mL of 1N sulfuric acid in a polypropylene centrifuge tube and mixing thoroughly. The sample was further diluted with 1 g of water and mixed again. From this mixture a 1 mL aliquot was taken and mixed with 1 mL of TISAB II solution in a fresh polypropylene centrifuge tube and mixed thoroughly prior to fluoride ion measurement. An average of 3 independent fluoride measurements were used to determine the fluoride concentration of each sample using the fluoride specific electrode and meter described above. Similar measurements were taken every 24 hours. The results are summarized in Table 8 below after 0 and 48 hours.

TABLE 8
Average fluoride ion concentration in polyol/amine
catalyst/foam additive/cyclopentane sample mixtures
after aging at room temperature

| | [F—] at time 0 | [F—] at 48 hours |
|---|---|---|
| | (ppm) | (ppm) |
| CE9 | 0.83 | 0.71 |
| CE10 | 1.51 | 168.42 |
| Example 3 | 11.74 | 10.67 |

The results illustrate that fluoride levels remain essentially unchanged over time for Example 3 and CE9, indicating little or no reaction of these foam additives with the polyol/amine catalyst mixture. However, CE10 reacts rapidly with the polyol/amine catalyst mixture resulting in a steep rise in fluoride ion levels over 48 hours. Thus, the use of Example 3 as a foam additive provides stability advantages vs. the commercial foam additive CE10 and provides much lower GWP and improved environmental sustainability vs. the PFC foam additive, CE9 (GWP=9000, 100 yr ITH). The relatively high stability of Example 3 towards the polyol/amine/foam blowing agent mixture is surprising in light of the reported susceptibility of perfluoroalkylsulfones to nucleophilic attack, including reactions with alcohols and amines, as described in J. Fluorine Chemistry, 117, 2002, pp 13-16.

### Battery Immersion Thermal Runaway Protection Performance

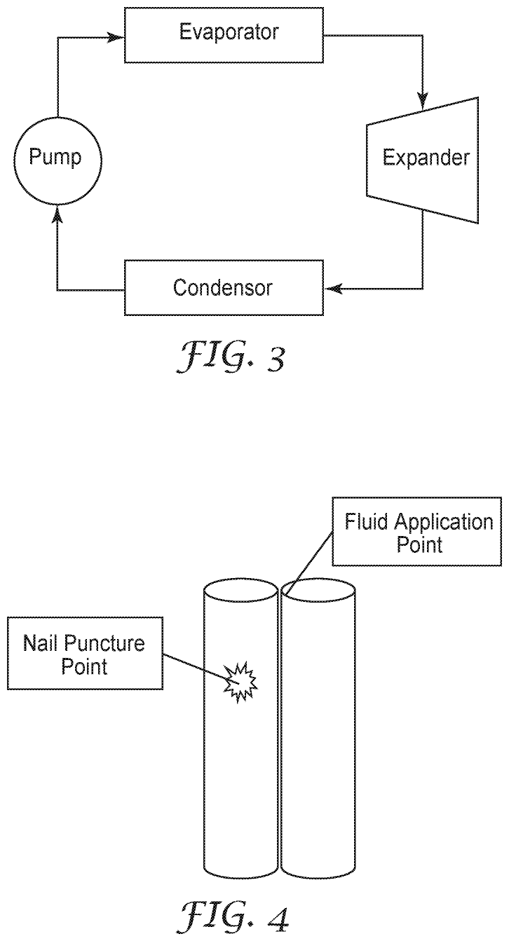

The following experiment was conducted to evaluate the effectiveness of exemplary fluids in mitigating cell-to-cell cascading thermal runaway. Two 3.5 amp-hour Graphite/NMC 18650 cells were welded together in a 2P configuration and charged to 100% SOC. Then, one of the cells was then driven into thermal runaway via nail puncture. After the initial event, fluid was applied between the two cells at various rates. FIG. 4 shows the nail and fluid application points. After fluid application, the adjacent cell's temperature was monitored to see if cascading thermal runaway occurred. Two different fluids were evaluated at two flow rates (25 ml/min for two minutes and 50 ml/min for one minute) and their relative effectiveness compared. The test fluids used were Example 3 (perfluorodiethylsulfone) and CE11 (NOVEC 649, a fluorinated ketone available from 3M Company, St. Paul, Minn., US), which has previously been disclosed as having utility in this application.

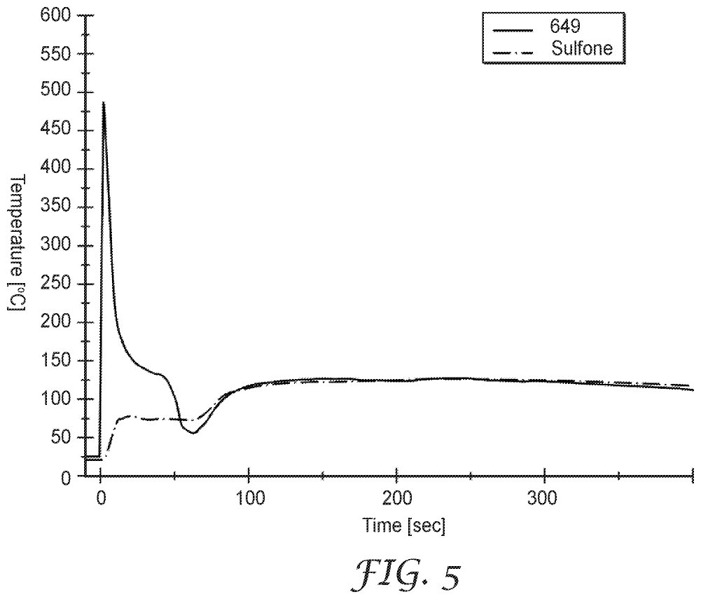

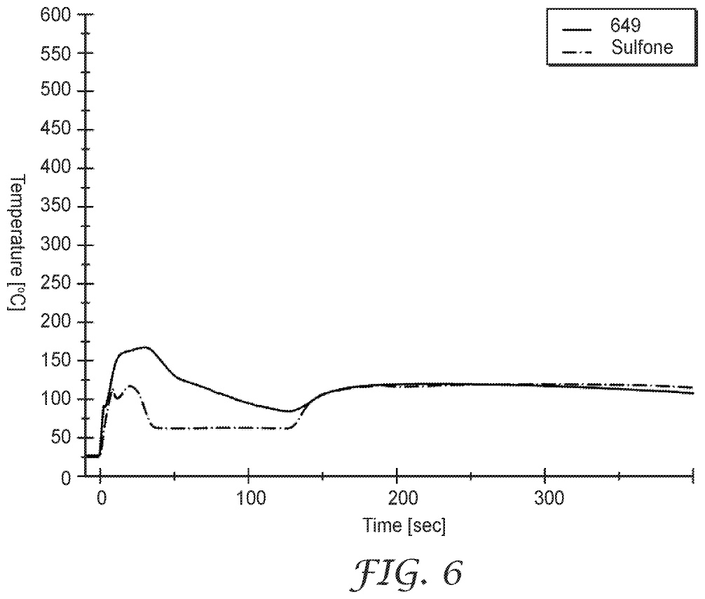

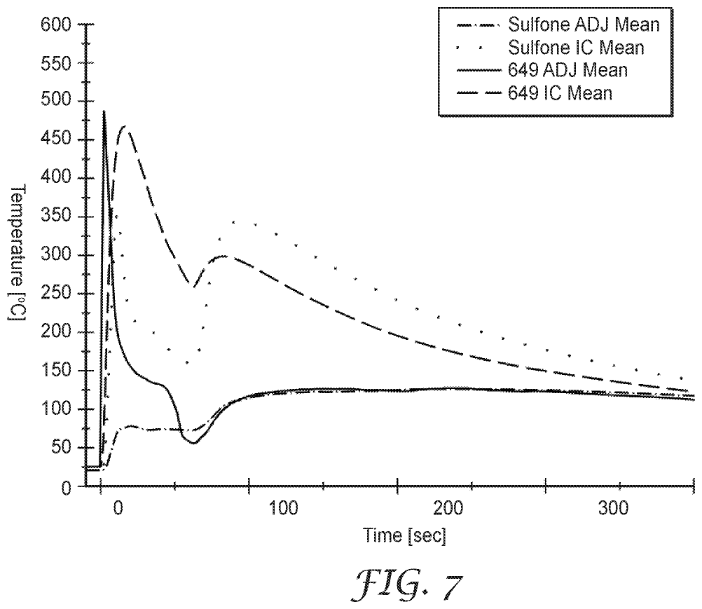

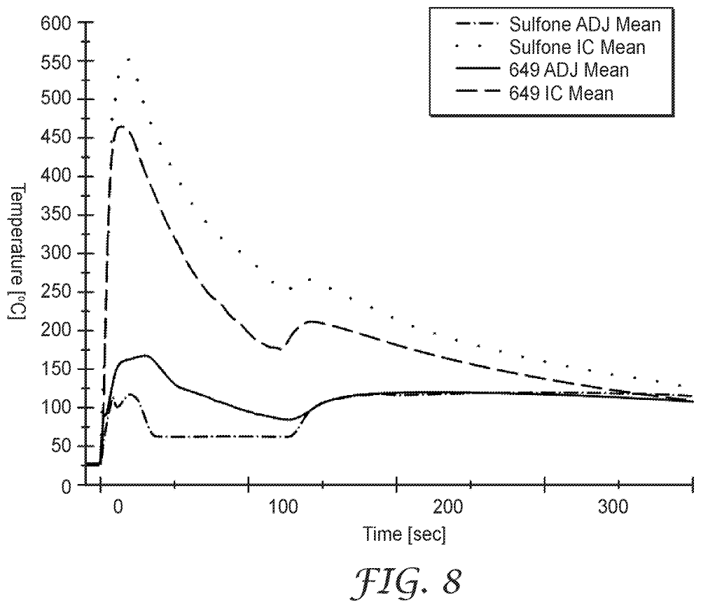

The mean temperatures in the adjacent cells are shown in FIGS. 5 and 6 for each of the flow rates. At both flow rates, Example 3 exhibited more effective temperature reduction than CE11 in the adjacent cell. FIGS. 7 and 8 compare the initial and adjacent cell temperatures when using Example 3 and CE11 at both flow rates. Example 3 was more effective than CE11 in reducing the temperature of the adjacent cell during fluid application, but cell temperatures increased to nearly identical levels once fluid was no longer being applied.

### Preparation of Polyurethane Foam

Example 3 (perfluorodiethylsulfone, 0.5 grams) was mixed into 5.8 g of cyclopentane to form a clear solution. This mixture was then added to 39.5 g of a polyether polyol resin with a viscosity of approximately 2000 cP at 25° C. (available from BASF, Ludwigshafen, Germany, under the trade name ELASTAPOR) and mixed for 30 seconds using a vortex mixer until an opaque emulsion had formed. The polyol resin contained a surfactant for foam stabilization and tertiary amine catalysts. To this emulsion, 54.2 grams of polymeric MDI isocyanate resin (LUPRANATE 277 from BASF) with a viscosity of approximately 350 cP at 25° C. was added while mixing at 4000 rpm for 15 seconds. The resulting mixture generated a free-rise foam that cured into a rigid, closed-cell foam with a density of approximately 30 kg/m³. A comparative example (CE12) was prepared using the same procedure, but omitting Example 3.

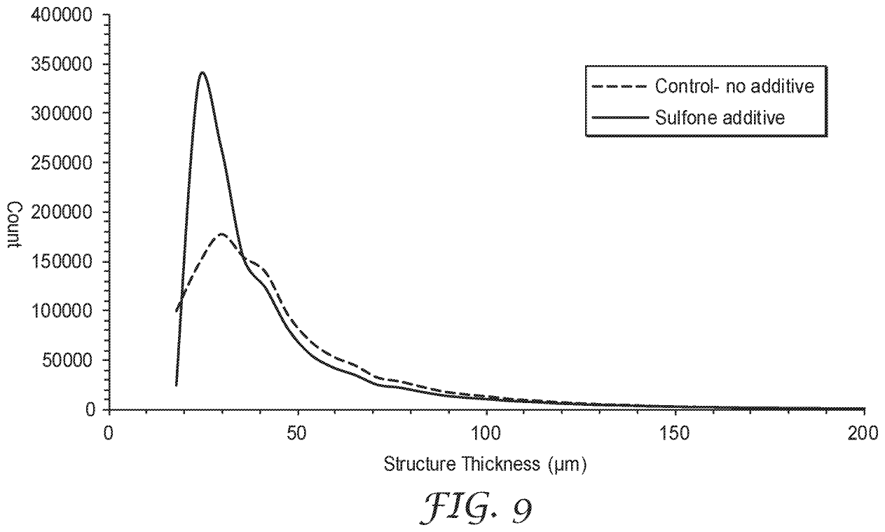

Samples of each foam were analyzed by X-ray microtomography to determine the size of the cells. A strip cut from each foam sample was scanned at 2.96 μm resolution. The resulting cell size distributions are plotted in FIG. 9 and summarized in Table 9. The foam produced using Example 3 as an additive displayed smaller cell diameters. Smaller cell sizes generally equate to better insulating properties in closed cell foams.

TABLE 9
Foam cell size distributions

| | Foam Prepared Using | Foam Prepared Using |
|---|---|---|
| | CE12 | Example 3 |
| number average cell | 46.3 | 43.8 |
| diameter (μm) | | |
| peak cell diameter (μm) | 29.6 | 23.7 |

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

1. A foamable composition comprising:
a blowing agent;
a foamable polymer or a precursor composition thereof; and
a nucleating agent, wherein said nucleating agent comprises a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

2. (canceled)

3. (canceled)

4. The foamable composition according to claim 1 wherein the blowing agent comprises an aliphatic hydrocarbon having from about 5 to about 7 carbon atoms, a cycloaliphatic hydrocarbon having from about 5 to about 7 carbon atoms, a hydrocarbon ester, water, or combinations thereof.

5. (canceled)

6. A foam made with the foamable composition according to claim 1.

7. (canceled)

8. A device comprising:
a dielectric fluid comprising a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1;
wherein the device is an electrical device and wherein the dielectric fluid further comprises a second dielectric fluid, wherein the second dielectric fluid comprises heptafluoroisobutyronitrile, 2,3,3,3-tetrafluoro-2-(trifluoromethoxy)propanenitrile, 1,1,1,3,4,4,4-heptafluoro-3-(trifluoromethyl)butan-2-one, or combinations thereof.

9. The device of claim 8, wherein said electrical device comprises gas-insulated circuit breakers, current-interruption equipment, a gas-insulated transmission line, gas-insulated transformers, or a gas-insulated substation.

10-12. (canceled)

13. The device according to claim 8, wherein R¹, R², and R³ are perfluorinated.

14. The device according to claim 8, wherein, n=0 and R¹ and R² are each independently a fluoroalkyl group having from 1 to 2 carbon atoms

15. (canceled)

16. An apparatus for converting thermal energy into mechanical energy in a Rankine cycle comprising:
a working fluid;
a heat source to vaporize the working fluid and form a vaporized working fluid;
a turbine through which the vaporized working fluid is passed thereby converting thermal energy into mechanical energy;
a condenser to cool the vaporized working fluid after it is passed through the turbine; and
a pump to recirculate the working fluid,
wherein the working fluid comprises a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

17. The apparatus according to claim 16, wherein the compound is present in the working fluid at an amount of at least 25% by weight based on the total weight of the working fluid.

18. The apparatus according to claim 16, wherein R¹, R², and R³ are perfluorinated.

19-21. (canceled)

22. An immersion cooling system comprising:
a housing having an interior space;
a heat-generating component disposed within the interior space; and
a working fluid liquid disposed within the interior space such that the heat-generating component is in contact with the working fluid liquid;
wherein the working fluid comprises a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

23. The system according to claim 22, wherein the compound is present in the working fluid at an amount of at least 25% by weight based on the total weight of the working fluid.

24. The system according to claim 22, wherein R¹, R², and R³ are perfluorinated.

25. The system according to claim 22, wherein the heat-generating component comprises an electronic device.

26. The system according to claim 22, wherein the electronic device comprises a computer server.

27. The system of claim 26, wherein the computer server operates at frequency of greater than 3 GHz.

28-33. (canceled)

34. A thermal management system for a lithium-ion battery pack comprising:
a lithium-ion battery pack; and
a working fluid in thermal communication with the lithium-ion battery pack;
wherein the working fluid comprises a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

35-37. (canceled)

38. A thermal management system for an electronic device, the system comprising:
an electronic device selected from a microprocessor, a semiconductor wafer used to manufacture a semiconductor device, a power control semiconductor, an electrochemical cell, an electrical distribution switch gear, a power transformer, a circuit board, a multi-chip module, a packaged or unpackaged semiconductor device, a fuel cell, or a laser; and
a working fluid in thermal communication with the electronic device;
wherein the working fluid comprises a compound having structural formula (I)
R¹SO₂R²(SO₂R³)ₙ (I)
where R¹, R², and R³ are each independently a fluoroalkyl group having from 1 to 10 carbon atoms that is linear, branched, or cyclic and optionally contain at least one catenated ether oxygen atom or a trivalent nitrogen atom, and n is 0 or 1.

39. The thermal management system according to claim 38, wherein the device is selected from a microprocessor, a semiconductor wafer used to manufacture a semiconductor device, a power control semiconductor, a circuit board, a multi-chip module, or a packaged or unpackaged semiconductor device.

40. The thermal management system according to claim 38, wherein the electronic device is at least partially immersed in the working fluid.

41-44. (canceled)
